SEQUENCE LISTING (16 sequences)

SEQ ID NO: 1 - protein, 360 aa, Homo sapiens
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 2 - protein, 8 aa, Artificial Sequence
  FLAG peptide
DYKDDDDK

SEQ ID NO: 3 - protein, 6 aa, Artificial Sequence
  Hemagglutinin peptide
YPYDVP

SEQ ID NO: 4 - protein, 10 aa, Artificial Sequence
  proto-oncoprotein myc tag
ILKKATAYIL

SEQ ID NO: 5 - protein, 10 aa, Artificial Sequence
  proto-oncoprotein myc tag
EQKLISEEDL

SEQ ID NO: 6 - DNA, 735 bp, Artificial Sequence
  codon-optimized cDNA encoding an anti-human CCR4 scFv
gacattgagttgactcaatctccatcttccttggctgtttctgctggtgagaaggttactatgtcttgtaagtcttcccaatctattttgtactcttccaaccaaaagaactacttggcttggtaccaacaaaagccaggtcaatctccaaagttgttgatttactgggcttctactagagagtctggtgttccagacagattcactggttctggttctggtactgacttcactttgactatttcttccgttcaagctgaggacttggctgtttactactgtcaccaatacttgtcttcctacactttcggtggtggtactaagttggagattaagggtggtggtggttctggtggtggtggatctggtggtggtggttctcaagttcaattgcaacaatctggtccagagttggttagaccaggtgcttctgttagaatttcttgtaaggcttctggttacactttcgcttcttactacattcaatggatgaagcaaagaccaggtcaaggtttggagtggattggttggattaacccaggtaacgttaacactaagtacaacgagaagttcaagggtaaggctactttgactgctgacaagtcttccactaccgcttacatgcaattgtcttccttgacttctgaggactctgctgtttacttctgtgctagatccacttactacagaccattggactactggggtcaaggtactaccgttactgtttcttcc

SEQ ID NO: 7 - protein, 245 aa, Artificial Sequence
  anti-human CCR4 scFv
DIELTQSPSSLAVSAGEKVTMSCKSSQSILYSSNQKNYLAWYQQKPGQSPKLLIYWASTRESGVPDRFTGSGSGTDFTLTISSVQAEDLAVYYCHQYLSSYTFGGGTKLEIKGGGGSGGGGSGGGGSQVQLQQSGPELVRPGASVRISCKASGYTFASYYIQWMKQRPGQGLEWIGWINPGNVNTKYNEKFKGKATLTADKSSTTAYMQLSSLTSEDSAVYFCARSTYYRPLDYWGQGTTVTVSS

SEQ ID NO: 8 - DNA, 705 bp, Artificial Sequence
  codon-optimized cDNA encoding an anti-human CCR4 scFv
gacattgagttgactcaatctccatcttccttggctgtttctgctggtgagaaggttactatgtcttgtaagtcttcccaatctattttgtactcttccaaccaaaagaactacttggcttggtaccaacaaaagccaggtcaatctccaaagttgttgatttactgggcttctactagagagtctggtgttccagacagattcactggttctggttctggtactgacttcactttgactatttcttccgttcaagctgaggacttggctgtttactactgtcaccaatacttgtcttcctacactttcggtggtggtactaagttggagattaagggtggtggtggatctcaagttcaattgcaacaatctggtccagagttggttagaccaggtgcttctgttagaatttcttgtaaggcttctggttacactttcgcttcttactacattcaatggatgaagcaaagaccaggtcaaggtttggagtggattggttggattaacccaggtaacgttaacactaagtacaacgagaagttcaagggtaaggctactttgactgctgacaagtcttccactaccgcttacatgcaattgtcttccttgacttctgaggactctgctgtttacttctgtgctagatccacttactacagaccattggactactggggtcaaggtactaccgttactgtttcttcc

SEQ ID NO: 9 - protein, 235 aa, Artificial Sequence
  anti-human CCR4 scFv
DIELTQSPSSLAVSAGEKVTMSCKSSQSILYSSNQKNYLAWYQQKPGQSPKLLIYWASTRESGVPDRFTGSGSGTDFTLTISSVQAEDLAVYYCHQYLSSYTFGGGTKLEIKGGGGSQVQLQQSGPELVRPGASVRISCKASGYTFASYYIQWMKQRPGQGLEWIGWINPGNVNTKYNEKFKGKATLTADKSSTTAYMQLSSLTSEDSAVYFCARSTYYRPLDYWGQGTTVTVSS

SEQ ID NO: 10 - DNA, 54 bp, Artificial Sequence
  synthetically generated primer
ccgctcgagccatggggtggtggtggttctgacattgagttgactcaatctcca

SEQ ID NO: 11 - DNA, 66 bp, Artificial Sequence
  synthetically generated primer
ccggaattccgccgcggatccaccaccaccagaaccaccaccaccggaagaaacagtaacggtagt

SEQ ID NO: 12 - DNA, 54 bp, Artificial Sequence
  synthetically generated primer
ccgctcgagggatccggtggtggtggttctgacattgagttgactcaatctcca

SEQ ID NO: 13 - DNA, 51 bp, Artificial Sequence
  synthetically generated primer
ccggaattcttagtggtggtggtggtggtgggaagaaacagtaacggtagt

SEQ ID NO: 14 - DNA, 41 bp, Artificial Sequence
  synthetically generated primer
cgcggatccaccaccacccttaatctccaacttagtaccac

SEQ ID NO: 15 - DNA, 32 bp, Artificial Sequence
  synthetically generated primer
ggaagatctcaagttcaattgcaacaatctgg

SEQ ID NO: 16 - protein, 5 aa, Artificial Sequence
  linker
GGGGS

(19) United States
(12) Patent Application Publication
(10) Pub. No.: US 2017/0121419 A1
(43) Pub. Date: May 4, 2017

(54) Anti-human Chemokine (C-C motif) Receptor 4 Immunotoxins

(71) Applicant: The General Hospital Corporation, Boston, MA (US)

(72) Inventors: Zhirui Wang, Malden, MA (US); David H. Sachs, Newton, MA (US); Christene A. Huang, Dover, MA (US); Joren Christian Madsen, Boston, MA (US)

(21) Appl. No.: 15/317,745

(22) PCT Filed: Jun. 12, 2015
(86) PCT No.: PCT/US15/35550

§ 371 Date: Dec. 9, 2016

Related U.S. Application Data
(60) Provisional application No. 62/011,235, filed on Jun. 12, 2014.
(60) Provisional application No. 62/049,096, filed on Sep. 11, 2014.

(51) Int. Cl. C07K 16/30; C07K 16/28

(52) U.S. Cl. CPC C07K 16/30; C07K 16/3061; C07K 16/2866; C07K 2317/56; A61K 48/00

(57) ABSTRACT

Anti-human chemokine (C—C motif) Receptor 4 immunotoxins and methods of use thereof, e.g., for depleting Tregs as an immunotherapy for the treatment of cancer; for the treatment of cancers associated with CCR4+ tumor cells such as skin homing cutaneous T cell lymphoma, adult T cell leukemia/lymphoma, and acute T-cell lymphoblastic leukemia; and for the depletion of CCCR4+ Th2 cells for the treatment of allergy-related conditions such as asthma.

### CLAIM OF PRIORITY

This application claims the benefit of U.S. Provisional Patent Application Ser. No. 62/011,235, filed on Jun. 12, 2014, and 62/049,096, filed on Sep. 11, 2014. The entire contents of the foregoing are hereby incorporated by reference.

### TECHNICAL FIELD

This invention relates to anti-human chemokine (C—C motif) Receptor 4 immunotoxins, and methods of use thereof.

### BACKGROUND

Regulatory T cells (Tregs) have been recognized as an important subset of T cells, and modulation of Tregs has been used in transplantation tolerance induction, autoimmune disease treatment, and cancer treatment. Antigen-specific immune responses such as those targeted against tumors are suppressed by Tregs characterized by CD4+CD25^{high}FoxP3+ expression. Treg depletion combined with tumor vaccination is a potentially promising approach to improve cancer treatment.

### SUMMARY

At least in part, the present invention is based on the discovery that three versions of anti-human CCR4 immunotoxins (monovalent, divalent and single chain foldback diabody) deplete FOXP3^{hi}CD45RA⁻CD25^{hi}Tregs. In in vitro protein synthesis inhibition assays, cell proliferation assays and flow cytometry antibody binding assays, all three versions of the toxins were shown to effectively deplete CCR4⁺ cells; the divalent toxin was shown to be more effective than the monovalent toxin and the diabody toxin was shown to be the most effective. These recombinant proteins can be used, e.g., for in vivo T-reg depletion to relieve repression of anti-tumor immune responses to treat cancer, and as a research tool to study immune regulation, tolerance induction, and autoimmune disease.

Thus, in a first aspect, the invention provides monovalent, bivalent and single chain fold back diabody anti-human CCR4 immunotoxins comprising a first part comprising a cytotoxic protein, and a second part comprising at least one anti-human chemokine (C—C motif) receptor 4 (CCR4) single chain variable fragment (scFv) sequence, e.g., two anti-human CCR4 scFv sequences comprising amino acids 1-245 of SEQ ID NO:7, optionally with one or both of a linker between the two anti-human CCR4 sequences, and a linker between the first and second parts. In some embodiments, the second part of the immunotoxin comprises SEQ ID NO:7. In some embodiments, the second part of the immunotoxin is at least 80%, 90%, 95%, or 99% identical to SEQ ID NO:7; such an immunotoxin that is at least 80% identical to SEQ ID NO:7 will retain the ability to bind CCR4⁺ cells and reduce protein synthesis and/or cell proliferation using an assay as described herein.

In some embodiments, the cytotoxic protein comprises diphtheria toxin, Pseudomonas exotoxin, or cytotoxic portions or variants thereof.

In some embodiments, the immunotoxins include a linker between the first and second parts.

In another aspect, the invention provides nucleic acid molecules, e.g., codon-optimized nucleic acid molecules (e.g., optimized for expression in a methylotropic yeast, e.g., of the species Pichia Pastoris), that encode the immunotoxins described herein, as well as vectors comprising the nucleic acid molecules, and host cells comprising and/or expressing the nucleic acid molecules.

In some embodiments, the host cell is a methylotropic yeast.

In some embodiments, the host cell is a cell of the species Pichia Pastoris.

In another aspect, the invention provides pharmaceutical compositions comprising the immunotoxins described herein, and a physiologically acceptable carrier.

In a further aspect, the invention provides methods for treating a subject who has a cancer, the method comprising administering to the subject a therapeutically effective amount of an immunotoxin described herein.

In some embodiments, the cancer comprises cancer cells that express CCR4, e.g., is selected from the group consisting of skin homing cutaneous T cell lymphoma, adult T cell leukemia/lymphoma, acute T-cell lymphoblastic leukemia, cutaneous T cell lymphoma/leukemia, anaplastic large cell lymphoma, peripheral T cell lymphoma; and adult T-cell leukemia/lymphoma. In some embodiments, the cancer is any cancer associated with the presence of Tregs, e.g., a solid tumor or other malignancy. In a further aspect, the invention provides methods for treating a subject who has an allergic disease (e.g., asthma, rhinitis, food allergy, and eczema), the method comprising administering to the subject a therapeutically effective amount of an immunotoxin described herein.

In some embodiments, the allergic disease is associated with the presence of T-helper type 2 (Th2) cells or invariant natural killer (iNKT) cells that express CCR4, e.g., allergic inflammation caused by an excess of Th2 cells that express CCR4.

In some embodiments, the methods include administering an immunotherapy to the subject. In some embodiments, the immunotherapy comprises administration of one or more of: dendritic cells or peptides with adjuvant; DNA-based vaccines; cytokines (e.g., IL-2); cyclophosphamide; anti-interleukin-2R immunotoxins; antibodies; virus-based vaccines (e.g., adenovirus); formulations of Toll-like Receptor or RIG-I-like receptor ligands; or adoptive T cell therapy or other cell therapy.

Also provided herein are methods for depleting CCR4-expressing FOXP3^{hi}CD45RA⁻CD25^{hi} regulatory T cells in a subject. The methods include administering to the subject an effective amount of an immunotoxin as described herein, or a nucleic acid encoding the immunotoxin.

In some embodiments, the subject has cancer or is an experimental model of autoimmune disease or transplant rejection.

In a further aspect, the invention provides methods for producing monovalent, divalent, or diabody anti-human CCR4 immunotoxins. The methods include expressing a nucleic acid molecule, e.g., a codon-optimized nucleic acid molecule, encoding an immunotoxin described herein in a host cell, e.g., in a methylotropic yeast; and substantially purifying the immunotoxin, thereby producing the composition. In some embodiments, the methylotropic yeast is of the species Pichia Pastoris.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Schematic diagrams of the monovalent, bivalent and single-chain fold-back diabody anti-human CCR4 immunotoxin.

FIG. 2. Codon-optimized anti-human CCR4 scFv (1567) DNA and amino acid sequence.

FIGS. 3A-F. SDS PAGE, Western blot and HPLC analysis of the anti-human CCR4 immunotoxins. A) SDS PAGE analysis (4-12% NuPAGE, Invitrogen); B) Western blot analysis using a mouse anti-His mAb (clone#: 4A12E4, Invitrogen); C) Western blot analysis using a mouse anti-diphtheria toxin mAb (clone#3B6, Meridian). Lane 1: Protein marker; Lane 2-3: monovalent anti-human CCR4 immunotoxin [DT390-scFv(1567), 70.26 kDa]; Lane 4-5: Bivalent anti-human CCR4 immunotoxin [DT390-BiscFv(1567), 97.57 kDa]; Lane 5-6: single-chain foldback diabody anti-human CCR4 immunotoxin (96.31 kDa). D-F) HPLC analysis with Shimadzu HPLC system using Superdex 200 size-exclusion column, 10/300 GL (GE healthcare, Cat#: 17-5175-01): D) monovalent anti-human CCR4 immunotoxin [DT390-scFv(1567)]; E) bivalent anti-human CCR4 immunotoxin [DT390-BiscFv(1567)]; F) single-chain fold-back diabody anti-human CCR4 immunotoxin.

FIGS. 4A-B. A) Binding analysis using flow cytometry of biotinylated monovalent anti-human CCR4 immunotoxin [DT390-scFv(1567)] (left panel), bivalent anti-human CCR4 immunotoxin [DT390-BiscFv(1567)] (middle panel), single-chain fold-back diabody anti-human CCR4 immunotoxin (right panel) to human CCR4+ CCRF-CEM cells (acute lymphoblastic leukemia cell line). Cells incubated with only the secondary staining (PE-conjugated streptavidin) served as a negative control and human CCR4 fluorescein mAb (clone#205410, R&D systems, cat# FAB1567F) for the positive control, mouse IgG2B fluorescein for the isotype control. Biotin-labeled porcine CD3-εγ (Peraino et al., 2012) was included as a negative control for background due to protein biotinylation. The data are representative of three individual experiments. B) KD Determination Using Flow Cytometry and Nonlinear Least Squares Fit. MFI was plotted over a wide range of concentrations of biotinylated 1) DT390-scFv(1567); 2) DT390-BiscFv(1567) and 3) single-chain fold-back diabody anti-human CCR4 immunotoxin. The accompanying least-squares fits are shown based on the hyperbolic equation y=m1+m2*m0/(m3+m0) where y=MFI at the given biotinylated anti-human CCR4 immunotoxin concentration, m0=biotinylated anti-human CCR4 immunotoxin concentration, m1=MFI of zero biotinylated anti-human CCR4 immunotoxin control, m2=MFI at saturation and m3=KD. A fitted KD of 5.66 nM was obtained for DT390-scFv(1567), 1.67 nM for DT390-BiscFv(1567) and 0.74 nM for single-chain fold-back diabody anti-human CCR4 immunotoxin.

FIGS. 5A-B. Blocking analysis of the anti-human CCR4 immunotoxins for the human CCR4 receptor on human CCR4+ CCRF-CEM leukemia cells. A) Flow cytometry histogram: unlabeled anti-human CCR4 immunotoxins were each incubated with CCRF-CEM cells at a range of concentrations for 15 minutes at 4° C. in the dark. Subsequently, without washing the cells, anti-human CCR4 mAb 1567 was added to each tube containing cells in the presence of the unlabeled immunotoxins. Binding affinity of the anti-human CCR4 immunotoxins to the human CCR4 receptor on CCRF-CEM cells was measured by a decrease in anti-human CCR4 mAb staining in the presence of increasing concentrations of the unlabeled immunotoxins. Murine IgG2B fluorescein was included as an isotype control. B) Blocking rate (%) was plotted versus the concentration of the binding competitor (monovalent, bivalent or single-chain fold-back diabody anti-human CCR4 immunotoxin). The relative binding affinity for any two competitors can be estimated from the ratio of their concentration at equal inhibition rate values or parallel curve regions. X-axis: immunotoxin concentration; Y-axis: blocking rate=(MFI value of the positive control−MFI value of the sample)/(MFI value of the positive control−MFI value of the isotype control)×100%. P<0.0001 by two-way ANOVA (n=3). Error bars indicate ±SD.

FIGS. 6A-D. A) Anti-human CCR4 immunotoxin-mediated protein synthesis inhibition in human CCR4+ CCRF-CEM cells in vitro: 1) monovalent anti-human CCR4 immunotoxin [DT390-scFv(1567), red line]; 2) bivalent anti-human CCR4 immunotoxin [DT390-BiscFv(1567), green line]; 3) single-chain fold-back diabody anti-human CCR4 immunotoxin (orange line); 4) DT390 alone. Y-axis: inhibition rate of the protein synthesis via the cpm value measuring incorporation of tritiated leucine. X-axis: plated anti-human CCR4 immunotoxin concentration. Cycloheximide (1.25 mg/mL) was used as a positive control. The negative control contained cells without immunotoxin. P<0.0001 by two-way ANOVA (n=3). Error bars indicate ±SD. B-D) Binding specificity analysis of the anti-human CCR4 immunotoxin to the target human CCR4+ CCRF-CEM cells in this in vitro protein synthesis inhibition assay using BiscFv(1567)-human Fc (1.06×10-7 M) as inhibitor: B) Monovalent anti-human CCR4 immunotoxin [DT390-scFv(1567)] with and without inhibitor; C) bivalent anti-human CCR4 immunotoxin [DT390-BiscFv(1567)] with and without inhibitor. D) single-chain fold-back diabody anti-human CCR4 immunotoxin with and without inhibitor. Y-axis: cpm value measuring incorporation of tritiated leucine. X-axis: plated anti-human CCR4 immunotoxin concentration. Wells containing the inhibitor were incubated for 1 hr at 37° C. before addition of the immunotoxin. Cycloheximide (1.25 mg/mL) was used as a positive control. Cells without immunotoxin served as the negative control. p<0.0001 by two-way ANOVA (n=3). Error bars indicate ±SD. Data are representative of multiple assays.

FIGS. 7A-D. A) Anti-human CCR4 immunotoxin-mediated cellular proliferation inhibition in human CCR4+ CCRF-CEM cells in vitro: 1) monovalent anti-human CCR4 immunotoxin [DT390-scFv(1567)]; 2) bivalent anti-human CCR4 immunotoxin [DT390-BiscFv(1567)]; 3) single-chain fold-back diabody anti-human CCR4 immunotoxin; 4) DT390. Y-axis: inhibition rate of the cell proliferation via the cpm value measuring incorporation of tritiated thymidine. X-axis: plated immunotoxin concentration. Cycloheximide (1.25 mg/mL) was used as a positive control. The negative control contained cells without immunotoxin. P<0.0001 by two-way ANOVA (n=3). Error bars indicate ±SD. B-D) Binding specificity analysis of the anti-human CCR4 immunotoxin to the target human CCR4+ CCRF-CEM cells during this in vitro cellular proliferation inhibition assay using BiscFv(1567)-human Fc (1.06×10-7 M) as inhibitor: B) Monovalent anti-human CCR4 immunotoxin [DT390-scFv(1567)] with (red) and without inhibitor; C) bivalent anti-human CCR4 immunotoxin [DT390-BiscFv(1567)] with and without inhibitor. D) single-chain fold-back diabody anti-human CCR4 immunotoxin with and without inhibitor. Y-axis: cpm value measuring incorporation of tritiated thymidine. X-axis: plated anti-human CCR4 immunotoxin concentration. Wells containing the inhibitor were incubated for 1 hr at 37° C. before addition of the immunotoxin. Cycloheximide (1.25 mg/mL) was used as a positive control. Cells without immunotoxin served as the negative control. p<0.0001 by two-way ANOVA (n=3). Error bars indicate ±SD. Data are representative of multiple assays.

FIGS. 8A-C. In vitro binding and depletion analysis of the anti-human CCR4 immunotoxins to human CCR4+ PBMC. A) Flow cytometry binding analysis of the anti-human CCR4 immunotoxins to the CCR4+ cells within human PBMC. Human PBMC was stained with the biotinylated anti-human CCR4 immunotoxin as primary staining and PE-conjugated streptavidin as second staining. First panel: monovalent anti-human CCR4 immunotoxin [DT390-scFv(1567)]; second panel: bivalent anti-human CCR4 immunotoxin [DT390-BiscFv(1567)]; third panel: single-chain foldback diabody anti-human CCR4 immunotoxin; fourth panel: anti-human CCR4 antibody [BiscFv(1567)-human Fc]. Human PBMC with only the secondary staining (PE-conjugated streptavidin) served as the negative control and human CCR4 fluorescein mAb (clone#205410, R&D systems, cat# FAB1567F) for the positive control, mouse IgG2B fluorescein for the isotype control. Biotin-labeled porcine CD3-εγ (Peraino et al., 2012) was included as a negative control for background due to protein biotinylation. The data are representative of three individual experiments. B) In vitro depletion of the CCR4+ cells within human PBMC using the anti-human CCR4 immunotoxins. Human PBMC was incubated with the unlabeled anti-human CCR4 immunotoxin at 37° C. for 48 h and analyzed by flow cytometry using biotinylated anti-human CCR4 antibody [BiscFv(1567)-human Fc] as primary staining and PE-conjugated streptavidin as second staining. Left panel: monovalent anti-human CCR4 immunotoxin [DT390-scFv(1567)]; middle panel: bivalent anti-human CCR4 immunotoxin [DT390-BiscFv(1567)]; right panel: single-chain foldback diabody anti-human CCR4 immunotoxin. Human PBMC with only the secondary staining (PE-conjugated streptavidin) served as the negative control and anti-human CCR4 antibody [BiscFv(1567)-human Fc] for the positive control. Biotin-labeled porcine CD3-εγ (Peraino et al., 2012) was included as a negative control for background due to protein biotinylation. The data are representative of three individual experiments. C) Flow cytometry binding analysis of the anti-human CCR4 immunotoxins to the Foxp3+CCR4+ human PBMC. Human PBMC was stained with Alexa Fluor® 647 anti-human Foxp3 mAb (clone#150D, Biolegend, cat#320014) and biotinylated anti-human CCR4 immunotoxin. First panel: monovalent anti-human CCR4 immunotoxin [DT390-scFv(1567)]; second panel: bivalent anti-human CCR4 immunotoxin [DT390-BiscFv(1567)]; third panel: single-chain foldback diabody anti-human CCR4 immunotoxin; fourth panel: anti-human CCR4 antibody [BiscFv(1567)-human Fc]. Human PBMC with only the secondary staining (PE-conjugated streptavidin) served as the negative control and anti-human CCR4 antibody [BiscFv(1567)-human Fc] for the CCR4 positive control, Alexa Fluor® 647 anti-human Foxp3 mAb for the Foxp3 positive control, Alex Fluor 647 Mouse IgG1 κ (clone# MOPC-21, Biolegend, cat#400136) for the isotype control of Alexa Fluor® 647 anti-human Foxp3 mAb. Biotin-labeled porcine CD3-εγ (Peraino et al., 2012) was included as a negative control for background due to protein biotinylation. The data are representative of three individual experiments.

FIG. 9. In vivo efficacy analysis of the anti-human CCR4 immunotoxins. NSG mice were IV injected with 1.0×107 human CCR4+ CCRF-CEM leukemia cells and treated from day 0 on with the anti-human CCR4 immunotoxin at 50 μg/kg BID for 4 consecutive days as one course, two course total and 3 day break between the two courses. 1) C21 immunotoxin control group (a non-related diphtheria toxin-based immunotoxin as negative control) (n=7, red curve) with a median survival time of 20 days; 2) monovalent anti-human CCR4 immunotoxin group (n=7, green curve) with a median survival time of 19 days; 3) bivalent anti-human CCR4 immunotoxin group (n=8, black curve) with a median survival time of 30 days; 4) single-chain foldback diabody anti-human CCR4 immunotoxin group (n=7, purple curve) with a median survival time of 32 days. The schedule of drug and tumor cell injection is pictured in the schematic below the survival curve. The vertical arrows indicate the days on which the tumor cells or anti-human CCR4 immunotoxins were injected.

FIGS. 10A-C In vitro binding and depletion analysis of the anti-human CCR4 immunotoxins to monkey CCR4+ PBMC. (A) Flow cytometry binding analysis of the anti-human CCR4 immunotoxins to CCR4+ cells within monkey PBMC. Monkey PBMC was stained with the biotinylated anti-human CCR4 immunotoxin as primary staining and PE-conjugated streptavidin as secondary staining. First panel: monovalent anti-human CCR4 immunotoxin [DT390-scFv (1567)]; second panel: bivalent anti-human CCR4 immunotoxin [DT390-BiscFv (1567)]; third panel: single-chain foldback diabody anti-human CCR4 immunotoxin; fourth panel: anti-human CCR4 antibody [BiscFv (1567)-human Fc]. Monkey PBMC with only the secondary staining (PE-conjugated streptavidin) served as the negative control and human CCR4 fluorescein mAb (clone#205410) as the positive control, mouse IgG2B fluorescein for the isotype control. Biotin-labeled porcine CD3-εγ 10 was included as a negative control for background due to protein biotinylation. (B) In vitro depletion of the CCR4+ cells within monkey PBMC using the anti-human CCR4 immunotoxins. Monkey PBMC was incubated with the unlabeled anti-human CCR4 immunotoxin at 37° C. for 48 h and analyzed by flow cytometry using biotinylated anti-human CCR4 antibody [BiscFv(1567)-human Fc] as primary staining and PE-conjugated streptavidin as secondary staining. Left panel: monovalent anti-human CCR4 immunotoxin [DT390-scFv (1567)]; middle panel: bivalent anti-human CCR4 immunotoxin [DT390-BiscFv (1567)]; right panel: single-chain foldback diabody anti-human CCR4 immunotoxin. Monkey PBMC with only the secondary staining (PE-conjugated streptavidin) served as the negative control and anti-human CCR4 antibody [BiscFv(1567)-human Fc] for the positive control. Biotin-labeled porcine CD3-εγ 10 was included as a negative control for background due to protein biotinylation. (C) Flow cytometry binding analysis of the anti-human CCR4 immunotoxins to the Foxp3+CCR4+ monkey PBMC. Monkey PBMC was stained with Alexa Fluor® 647 anti-human Foxp3 mAb (clone#150D) and biotinylated anti-human CCR4 immunotoxin. First panel: monovalent anti-human CCR4 immunotoxin [DT390-scFv(1567)]; second panel: bivalent anti-human CCR4 immunotoxin [DT390-BiscFv(1567)]; third panel: single-chain foldback diabody anti-human CCR4 immunotoxin; fourth panel: anti-human CCR4 antibody [BiscFv(1567)-human Fc]. Monkey PBMC with only the secondary staining (PE-conjugated streptavidin) served as the negative control and anti-human CCR4 antibody [BiscFv(1567)-human Fc] for the CCR4 positive control, Alexa Fluor® 647 anti-human Foxp3 mAb for the Foxp3 positive control, Alex Fluor 647 Mouse IgG1 κ (clone# MOPC-21) for the isotype control of Alexa Fluor® 647 anti-human Foxp3 mAb. Biotin-labeled porcine CD3-εγ 10 was included as a negative control for background due to protein biotinylation. All of the data (A-C) are representative of three individual experiments.

FIGS. 11A-H Monkey Treg depletion in the peripheral blood using the foldback diabody anti-human CCR4 immunotoxin. The immunotoxin was injected at 25 μg/kg IV, BID for four consecutive days. M1815 and M1915: two cyno monkeys. (A) The CCR4+ cell depletion in the peripheral blood was monitored by flow cytometry using the antibodies against human CD4 and CCR4 (CD4+CCR4+). (B) Representative flow cytometry analysis (day 0 to day 4) of the CCR4+ cell depletion in the peripheral blood using the antibodies against CD4 and CCR4 (CD4+CCR4+). (C) The CCR4+Foxp3+ Treg depletion in the peripheral blood was monitored by flow cytometry using the antibodies against human CCR4 and Foxp3 (CCR4+Foxp3+ among the gated CD4+ cells). (D) Representative flow cytometry analysis (day 0 to day 4) of the CCR4+Foxp3+ cell depletion in the peripheral blood using the antibodies against CCR4 and Foxp3 (CCR4+Foxp3+ among the gated CD4+ cells). (e-h) Off-target analysis of the depletion in the peripheral blood by flow cytometry: (E) The CD8+ T cells in the peripheral blood were monitored by flow cytometry using the antibodies against human CD3 and CD8 (CD3+CD8+). (F) The CD20+ B cells in the peripheral blood were monitored by flow cytometry using antibodies against human CD3 and CD20 (CD3-CD20+). (G) The CD4+ cells in the peripheral blood were monitored by flow cytometry using the antibodies against human CD3 and CD4 (CD3+CD4+). (H) The CD14+CD11b+ monocytes in the peripheral blood were monitored by flow cytometry using antibodies against human CD14 and CD11b (CD14+CD11b+).

FIGS. 12A-B Monkey Treg depletion in the lymph nodes using the flodback diabody anti-human CCR4 immunotoxin. The lymph node biopsies were performed before and after the immunotoxin treatment. (A) The lymph node Treg depletion was monitored by flow cytometry using the antibodies against CD4, CCR4, CD45RA and Foxp3 (CCR4+ cells: CD4+CCR4+, CCR4+ Tregs: CCR4+Foxp3+ among the gated CD4+ cells, effector Tregs: CD45RA-Foxp3+ among the gated CD4+ cells). The lymph node off-target depletion was monitored by flow cytometry using antibodies against CD3, CD4, CD8 and CD20 (CD4+ T cells: CD3+CD4+, CD8+ T cells: CD3+CD8+, CD20+ B cells: CD3-CD20+). (B) Representative flow cytometry analysis of the lymph node biopsy samples using antibodies against CD4, CCR4 and Foxp3 (CD4+CCR4+, CCR4+Foxp3+ among the gated CD4+ cells) before and after immunotoxin treatment.

### DETAILED DESCRIPTION

Regulatory T cells (Tregs) have been widely recognized as crucial players in controlling immune responses. Because their major role is to ensure that the immune system is not over reactive, Tregs have been the focus of multiple research studies including those investigating transplantation tolerance, autoimmunity and cancer treatment. An effective reagent capable of depleting Tregs in vivo would facilitate better cancer treatment and allow mechanistic studies of the role of Treg in transplantation tolerance and the development of autoimmune disease.

On their surface, FOXP3^{hi}CD45RA⁻CD25^{hi}Tregs constitutively express high levels of the chemokine (C—C motif) receptor 4 (CCR4) (Sugiyama et al., 2013). Described herein are novel monovalent, bivalent and single chain foldback diabody anti-human CCR4 immunotoxins and the functional activity of these reagents in vitro. As shown in Example 1, genetically linking two anti-human CCR4 scFv domains in tandem, thereby generating a bivalent immunotoxin, results in significantly improved capacity in targeting human CCR4⁺ cells in vitro. Furthermore forcing two anti-human CCR4 scFv domains to dimerize, thereby creating a single chain foldback diabody toxin, results in even greater capacity than the bivalent toxin in targeting human CCR4⁺ cells. Binding analysis by flow cytometry showed that the bivalent anti-human CCR4 immunotoxin has notably increased affinity for human CCR4⁺ cells than the monovalent version and the diabody anti-human CCR4 toxin has an even greater affinity for human CCR4⁺ cells. In vitro functional analysis demonstrated that the bivalent isoform has an increased potency of approximately 40 fold in inhibiting cellular proliferation and protein synthesis in human CCR4 cells compared to the monovalent anti-human CCR4 immunotoxin. The single chain foldback diabody anti-human CCR4 toxin has an increased potency of approximately 16 fold over the bivalent anti-human CCR4 toxin in both the cellular proliferation and protein synthesis assays. These results demonstrated that 1) the monovalent, bivalent and single chain foldback diabody anti-human CCR4 immunotoxins are capable of blocking the binding of an anti-human CCR4 monoclonal antibody to human CCR4 by flow cytometry; and 2) the bivalent anti-human CCR4 toxin is more efficient than the monovalent version and the single chain foldback diabody is more efficient than the bivalent version for depletion of Tregs.

The exemplary reagents constructed in this study were generated by genetically linking one or two anti-human CCR4 scFv polypeptides to a toxin, e.g., the truncated diphtheria toxin (DT390). Without wishing to be bound by theory, this reagent is believed to function by first binding to the cell surface via the anti-human CCR4/CCR4 receptor interaction, then the toxin, e.g., DT390 domain, is internalized followed by inhibition of protein synthesis resulting in cell death. Monovalent, bivalent, and single chain foldback diabody immunotoxins against human CCR4 were created. Human effector Tregs express CCR4 at high levels whereas naïve Tregs or Th1 and most other cells in the immune system, including CD8⁺ T cells, NK cells, CD14⁺ monocytes/macrophages, dendritic cells and B cells, express CCR4 at barely detectable levels (Sugiyama et al., 2013). For these reasons it is expected that targeting Treg through CCR4 will result in more specific effector Treg depletion.

Three versions of the anti-human CCR4 immunotoxin were designed for this study: 1) monovalent anti-human CCR4 immunotoxin; 2) divalent anti-human CCR4 immunotoxin; 3) single chain foldback diabody anti-human CCR4 immunotoxin. Using a human CCR4⁺ acute lymphoblastic leukemia cell line (CCRF-CEM), in vitro analysis of the immunotoxins' ability to inhibit protein synthesis demonstrated that all three toxins were effective but the single chain foldback diabody toxin was the most effective. These in vitro results are consistent with binding affinity as assessed by flow cytometry with the same cell line. The recombinant proteins described herein also have great potential as a useful tool for in vivo depletion of Tregs and human CCR4⁺ cancer cells.

The United States Federal Drug Administration-approved a truncated diphtheria toxin based human IL-2 immunotoxin, ONTAK (Denileukin diftitox, DAB389IL-2, Eisai Medical Research, Inc.) that has been shown to deplete Tregs in both pre-clinical and clinical settings thereby facilitating improved cancer treatment (Morse et al., Blood 112:610-618 (2008); Mahnke et al., Int. J. Cancer 120:2723-33. (2007); Litzinger, et al., Blood 110:3192-3201 (2007); Gritzapis et al., Cancer Immunol Immunother. 61:397-407 (2012)). Natural killer (NK) cells are a very important component of the innate immune system as their functions include fighting pathogenic infections and cancer (Salagianni et al., J. Immunol. 186:3327-35 (2011)). While it is somewhat effective in depleting Tregs during cancer treatment, ONTAK also creates unwanted side effects as it has been shown to completely deplete NK cells for a prolonged period in a cynomolgus monkey model (Yamada et al., J. Immunol. 188:6063-70 (2012)). However, this E. coli expressed, monovalent human IL-2 immunotoxin was unable to achieve optimal levels of Treg depletion (Morse et al., 2008; Barnett et al., Am. J Reprod Immunol 54, 369 (2005); Telang et al., BMC. Cancer 11, 515 (2011); Attia et al., J Immunother. 28, 582 (2005); Yamada et al., J Immunol 188, 6063 (2012)) and its production has been discontinued since 2011. Since then a more effective bivalent IL-2 toxin was discovered that showed less secondary effects than the monovalent version. Targeting CCR4 instead of CD25 should improve upon the targeted depletion of Tregs and leave NK cells undisturbed because of the very low expression levels of CCR4 in NK cells and the high levels of expression in effector Tregs (Yamada et al., J Immunol 188, 6063 (2012)).

Endotoxin is another common concern when using E. coli expression system. The present study utilized a diphtheria toxin-resistant yeast Pichia Pastoris expression system (Liu et al., Protein Expr Purif. 30, 262 (2003)), which offers greatly enhanced protein expression levels, purification and yield. Moreover, two anti-human CCR4 scFv domains were genetically linked to generate a bivalent immunotoxin, and in a third version the linker was shortened to produce a single chain foldback diabody immunotoxin with increased affinity for CCR4. The bivalent anti-human CCR4 immunotoxin showed significantly higher efficacy for human CCR4+ cells compared to the monovalent isoform and the single chain foldback diabody version showed an even higher efficacy. Linking two anti-human CCR4 domains in tandem may increase the immunotoxins' affinity for human CCR4, subsequently facilitating a more efficient internalization, and causing a notable increase in potency. Producing the recombinant anti-human CCR4 immunotoxins in yeast rather than E. coli and generating bivalent and diabody versions, augments the potential for clinical application of this reagent. Monovalent, bivalent, and single chain foldback diabody anti-human CCR4 immunotoxin reagents are available through our self-managed MGH-DF/HCC Recombinant Protein Expression and Purification Core facility for preclinical development and translational research.

CCR4

Chemokine (C—C motif) receptor 4 (CCR4) is a G protein coupled receptor. It is a receptor for the chemokines CCL2, CCL4, CCL5, CCL17, and CCL22. CCR4 is constitutively expressed on Tregs and has a very low expression in most other cells of the immune system (Sugiyama et al 2013). Some human cancers (e.g., adult T-cell leukemia/lymphoma (Ishida et al., Cancer Sci. 97(11): 1139-1146 (2006)); skin homing cutaneous T cell lymphoma (Ferenczi et al., J Invest Dermatol 119(6): 1405-1410 (2002)); acute T-cell lymphoblastic leukemia (Yoshie et al., Blood 99(5): (2002)); Cutaneous T cell lymphoma/leukemia (CTCL), anaplastic large cell lymphoma (ALCL), peripheral T cell lymphoma (PTCL); and adult T-cell leukemia/lymphoma (ATLL) Yoshie and Matsushima, Int Immunol. 2014 Aug. 2. pii: dxu079) have also demonstrated high levels of CCR4 expression and can also be treated using methods described herein.

The immunotoxins described herein comprise a mouse anti-human CCR4 scFv sequence, and preferably two anti-human CCR4 scFv sequences, optionally with a short intervening linker there between to enable both of the sequences to retain binding function. For example, all or part of the human CCR4 sequence can be used to generate anti-human CCR4 antibodies, e.g., as set forth at GenBank Acc. Nos. NM_005508.4 (nucleic acid) and NP_005499.1 (amino acid); that amino acid sequence is as follows:

1 mnptdiadtt ldesiysnyy lyesipkpct kegikafgel flpplyslvf vfgllgnsvv

61 vlvlfkykrl rsmtdvylln laisdllfvf slpfwgyyaa dqwvfglglc kmiswmylvg

121 fysgiffvml msidrylaiv havfslrart ltygvitsla twsvavfasl pgflfstcyt

181 ernhtycktk yslnsttwkv lssleinilg lviplgimlf cysmiirtlq hcknekknka

241 vkmifavvvl flgfwtpyni vlfletivel evlqdctfer yldyaiqate tlafvhccln

301 piiyfflgek frkyilqlfk tcrglfvlcq ycgllqiysa dtpsssytqs tmdhdlhdal (SEQ ID NO:1)

See, e.g., Williams et al., Protein Engineering 1(6):493-498, 1987; Foss, Ann. NY Acad Sci. 2001 September; 941:166-76; and Kelley et al., Proc. Natl. Acad. Sci. USA 85:3980-3984, 1988, all of which are incorporated by reference herein for their relevant teachings.

Codon optimization is desirable to express proteins such as immunotoxins in the Pichia Pastoris expression system (Woo et al., Protein Expr. Purif. 25, 270-282, 2002). A codon-optimized DT390 nucleotide sequence (Woo et al., 2002) was used for the DT390 domain. The DT390 has been modified to include an NH2 terminal alanine (A) and double mutations (dm) to prevent glycosylation in the eukaryotic expression system, Pichia Pastoris (Woo et al., 2002, Liu et al., Protein Expr. Purif. 19, 304-311, 2000; Liu et al., Protein Expr. Purif. 30, 262-274, 2003). The codon-optimized anti-human CCR4 nucleotide sequences described herein were used for the anti-human CCR4 domain.

In some embodiments, the mutation is a conservative substitution. Such changes include substituting any of isoleucine (I), valine (V), and leucine (L) for any other of these hydrophobic amino acids; aspartic acid (D) for glutamic acid (E) and vice versa; glutamine (Q) for asparagine (N) and vice versa; and serine (S) for threonine (T) and vice versa. Other substitutions can also be considered conservative, depending on the environment of the particular amino acid and its role in the three-dimensional structure of the protein. For example, glycine (G) and alanine (A) can frequently be interchangeable, as can alanine (A) and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the differing pK's of these two amino acid residues are not significant. Still other changes can be considered “conservative” in particular environments (see, e.g. Table III of US20110201052; pages 13-15 “Biochemistry” 2nd ED. Stryer ed (Stanford University); Henikoff et al., PNAS 1992 Vol 89 10915-10919; Lei et al., J Biol Chem 1995 May 19; 270(20):11882-6).

In some embodiments, the methods include introducing one or more additional mutations into the anti-human CCR4 scFv sequence. Thus, in some embodiments, the sequence can be at least 80%, 85%, 90%, 95%, or 99% identical to at least 60%, 70%, 80%, 90%, or 100% of an anti-human CCR4 scFv sequence, e.g., SEQ ID NO:7; e.g., the sequence can include 1, 2, 3, 4, 5, 6, 7, 8, 9, 10, 11, 12, 13, 14, 15, 16, 17, 18, 19, or 20 mutations. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is typically at least 80% of the length of the reference sequence, and in some embodiments is at least 90% or 100%. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. In another embodiment, the percent identity of two amino acid sequences can be assessed as a function of the conservation of amino acid residues within the same family of amino acids (e.g., positive charge, negative charge, polar and uncharged, hydrophobic) at corresponding positions in both amino acid sequences (e.g., the presence of an alanine residue in place of a valine residue at a specific position in both sequences shows a high level of conservation, but the presence of an arginine residue in place of an aspartate residue at a specific position in both sequences shows a low level of conservation).

For purposes of the present invention, the comparison of sequences and determination of percent identity between two sequences can be accomplished using a Blossum 62 scoring matrix with a gap penalty of 12, a gap extend penalty of 4, and a frameshift gap penalty of 5.

As noted above, the immunotoxins described herein include at least one anti-human CCR4 scFv sequence, preferably linked by a short intervening linker, e.g., 1-50 amino acids in length. The linker can have any composition so long as it (1) does not interfere with binding of the anti-human CCR4 to human CCR4; and (2) separates the two anti-human CCR4 sequences to avoid interference with each other (e.g., steric or other interference). Preferably the linker does not encode another protein. In some embodiments, the linker is comprised of serine, alanine and glycine residues, e.g., is at least 50% alanine, glycine, or serine. In some embodiments, the linker comprises one or more G₄S repeats, e.g., G₄S, (G₄S)₂, or (G₄S)₃.

The exemplary (G₄S)₃ linker used herein has been successfully used in following immunotoxins: anti-porcine CD3 immunotoxins (Wang et al, 2011, Bioconjug Chem. 22:2014-2020); anti-human CD3 immunotoxin (Woo et al. 2002, Protein Expr Purif. 25:270-282); anti-monkey CD3 immunotoxin (Kim et al., 2007, Protein Eng Des Sel. (PEDS). 20:425-432).

A linker as described herein may also be present between the anti-human CCR4 scFv and the toxin. A linker as described herein may also be present between the V_{H} and V_{L} regions of the anti-human CCR4 scFv sequence.

Single Chain Variable Fragment (scFv)

Single chain variable fragments are fusion proteins of the variable regions of the heavy (V_{H}) and light (V_{L}) chains of immunoglobulins, connected into a single polypeptide chain with a short linker peptide (e.g., 1-50 or 10-25 amino acids). The linker allows the scFv to fold into a structure suitable for antigen binding. A monovalent construct includes a single ScFv. Two ScFvs can be linked, e.g., using a linker of 1-50, e.g., 10-25, amino acids, to form a divalent construct. Such divalent ScFv fusions include two V_{H} and two V_{L} domains with flexible linkers in between (e.g., V_{H}-linker1-V_{L}-linker2-V_{H}-linker3-V_{L}). In some embodiments, linkers 1 and 3 are the same, and are 10-25 amino acids long, and linker 2 is 15-20. Methods for optimizing linkers are known in the art, see, e.g., Shan et al., J Immunol. 1999 Jun. 1; 162(11):6589-95. See also Ahmad et al., Clinical and Developmental Immunology, vol. 2012, Article ID 980250, 15 pages, 2012.

Single Chain Foldback Diabodies

A diabody refers to fusion proteins that are constructed with two ScFvs linked with a shortened linker (e.g., only 1 G₄S repeat or other linker with 5 or less amino acids) between the V_{L} and V_{H} portions of the scFv (Holliger et al., Proc Natl Acan Sci, 90:6444-6448 (1993)). The shortened linker does not allow the two variable regions of the peptide to fold together and the single chain antibody fragments are forced to dimerize. The result is an antibody with multiple antigen binding sites.

### Fusion Proteins

The recombinant anti-human CCR4 immunotoxin fusion proteins described herein include a cytotoxic non-anti-human CCR4 sequence fused to the N or C terminal (the fusion was at the N-terminal in this study) of a CCR4 binding domain, e.g., an antigen-binding portion of an anti-CCR4 antibody, e g., an ScFv comprising the V_{H} and V_{L} regions of a mouse anti-human CCR4 scFv antibody (e.g., Mab1567, clone 205410, R&D systems; see WO2009086514). In some embodiments, the cytotoxic non-anti-human CCR4 sequence is a cytotoxic protein, e.g., Idarubicin; CRM9 (e.g., FN18-CRM9, Knechtle et al., Transplantation 1997; 63:1-6); or pokeweed antiviral protein. In some embodiments, the cytotoxic protein is a bacterial toxin, e.g., diphtheria toxin (DT) or portions or variants thereof such as DT390, DT389, DT383, DT370 or other truncated mutants, e.g., Met1-Thr387, e.g., as described in Aullo et al., EMBO J. 11(2):575-83 (1992); Abi-Habib et al., Blood. 104(7):2143-2148 (2004); Perentesis et al., Proc. Nati. Acad. Sci. USA 85:8386-8390 (1988); Zettlemeissl et al., Gene. 41(1):103-111 (1986); US 2009/0010966; US20090041797; U.S. Pat. No. 5,843,711; U.S. Pat. No. 7,585,942; U.S. Pat. No. 7,696,338; or US20080166375; or Pseudomonas exotoxin (PE), or portions or variants thereof, e.g., as described in U.S. Pat. Nos. 4,545,985; 4,892,827; 5,458,878; 7,314,632; Song et al., Protein Expression and Purification 44(1):52-57 (2005); Theuer et al., J. Biol. Chem. 267(24):16872-16877 (1992); Heimbrook et al., Proc Natl Acad Sci USA. 87(12):4697-4701 (1990); Debinski et al., Mol Cell Biol. 11(3):1751-1753 (1991); Chaudhary et al., Proc. Nadl. Acad. Sci. USA 87:308-312 (1990). In some embodiments, the cytotoxic protein is a plant toxin, e.g., a plant holotoxin (e.g., class II ribosome-inactivating proteins such as ricin (e.g., deglycosylated ricin A chain (dgA)), abrin, mistletoe lectin, or modeccin) or hemitoxin (class I ribosome-inactivating proteins, e.g., PAP, saporin, bryodin 1, bouganin, or gelonin), or fragments or variants thereof that retain cytotoxic activity. See, e.g., Neville et al., J Contr Rel 1993; 24:133-141; Vallera, Blood 1994; 83:309-317; Vitetta et al., Immunology Today 1993; 14:252-259; Kreitman et al., AAPS Journal. 2006; 8(3):E532-E551). Suitable sequences are known in the art.

Peptide Tags

In some embodiments, the immunotoxins further include a peptide tag useful for purification. In some embodiments, the tag comprises histidines, e.g., two or more, e.g., three, four, five or six histidine residues at the C-terminus and purification is achieved by binding to a nickel or cobalt column. In some embodiments, the tag comprises glutathione-S-transferase (GST) and recovery is by affinity to substrate glutathione bound to a column, e.g., glutathione sepharose. In some embodiments, the tag comprises a FLAG peptide (e.g., N-DYKDDDDK-C (SEQ ID NO:2) or a variant thereof) and protein is recovered with specific antibody to the peptide. In some embodiments, the tag comprises an epitope derived from the Influenza protein hemagglutinin (HA) (e.g., N-YPYDVP-C(SEQ ID NO:3)) and protein is recovered using an anti-HA antibody that binds the epitope. In some embodiments, the tag comprises an epitope derived from the human proto-oncoprotein myc (e.g., N-ILKKATAYIL-C(SEQ ID NO:4), or N-EQKLISEEDL-C(SEQ ID NO:5)), and recovery is performed with an anti-myc antibody.

In some embodiments, the protein further comprises a proteolytic cleavage site between the purification tag and the CTLA-4 sequence, and after purification the protein is treated with the protease to remove the purification tag. Examples include the PreScission protease, thrombin, and factor Xa. Enterokinase sites that enable tag cleavage without leaving behind extra amino acids are preferred. In some embodiments, an exopeptidase is used to remove N-terminal His-tags (e.g., Qiagen TAGZyme). See, e.g., The Recombinant Protein Handbook, Protein Amplification and Simple Purification, Amersham Biosciences, available online at 130.15.90.245/methods/hand-books%20and%20manuals/the%20recombinant%20protein%20 handbook.pdf.

Codon Optimization

In addition, the nucleic acid sequences used in the present methods are preferably codon-optimized for expression in a selected expression system, e.g., in Pichia Pastoris (See, e.g., Woo et al., Protein Expr. Purif. 25, 270-282, 2002). In order to optimize expression in non-mammalian cells, codon optimization specific for a selected host organism can be used. For example, in embodiments where P. Pastoris is used as a host organism, the following Table 1 (source: kazusa.or.jp) can be used to select codons:

TABLE 1
Codon Optimization Table for Pichia Pastoris

| triplet | UUU | UCU | UAU | UGU |
|---|---|---|---|---|
| amino acid | F | S | Y | C |
| fraction | 0.54 | 0.29 | 0.47 | 0.64 |
| frequency: per 1000 | 24.1 | 24.4 | 16.0 | 7.7 |
| (number) | (1963) | (1983) | (1300) | (626) |
| triplet | UUC | UCC | UAC | UGC |
| amino acid | F | S | Y | C |
| fraction | 0.46 | 0.20 | 0.53 | 0.36 |
| frequency: per 1000 | 20.6 | 16.5 | 18.1 | 4.4 |
| (number) | (1675) | (1344) | (1473) | (356) |
| triplet | UUA | UCA | UAA | UGA |
| amino acid | L | S | * | * |
| fraction | 0.16 | 0.18 | 0.51 | 0.20 |
| frequency: per 1000 | 15.6 | 15.2 | 0.8 | 0.3 |
| (number) | (1265) | (1234) | (69) | (27) |
| triplet | UUG | UCG | UAG | UGG |
| amino acid | L | S | * | W |
| fraction | 0.33 | 0.09 | 0.29 | 1.00 |
| frequency: per 1000 | 31.5 | 7.4 | 0.5 | 10.3 |
| (number) | (2562) | (598) | (40) | (834) |
| triplet | CUU | CCU | CAU | CGU |
| amino acid | L | P | H | R |
| fraction | 0.16 | 0.35 | 0.57 | 0.17 |
| frequency: per 1000 | 15.9 | 15.8 | 11.8 | 6.9 |
| (number) | (1289) | (1282) | (960) | (564) |
| triplet | CUC | CCC | CAC | CGC |
| amino acid | L | P | H | R |
| fraction | 0.08 | 0.15 | 0.43 | 0.05 |
| frequency: per 1000 | 7.6 | 6.8 | 9.1 | 2.2 |
| (number) | (620) | (553) | (737) | (175) |
| triplet | CUA | CCA | CAA | CGA |
| amino acid | L | P | Q | R |
| fraction | 0.11 | 0.42 | 0.61 | 0.10 |
| frequency: per 1000 | 10.7 | 18.9 | 25.4 | 4.2 |
| (number) | (873) | (1540) | (2069) | (340) |
| triplet | CUG | CCG | CAG | CGG |
| amino acid | L | P | Q | R |
| fraction | 0.16 | 0.09 | 0.39 | 0.05 |
| frequency: per 1000 | 14.9 | 3.9 | 16.3 | 1.9 |
| (number) | (1215) | (320) | (1323) | (158) |
| triplet | AUU | ACU | AAU | AGU |
| amino acid | I | T | N | S |
| fraction | 0.50 | 0.40 | 0.48 | 0.15 |
| frequency: per 1000 | 31.1 | 22.4 | 25.1 | 12.5 |
| (number) | (2532) | (1820) | (2038) | (1020) |
| triplet | AUC | ACC | AAC | AGC |
| amino acid | I | T | N | S |
| fraction | 0.31 | 0.26 | 0.52 | 0.09 |
| frequency: per 1000 | 19.4 | 14.5 | 26.7 | 7.6 |
| (number) | (1580) | (1175) | (2168) | (621) |
| triplet | AUA | ACA | AAA | AGA |
| amino acid | I | T | K | R |
| fraction | 0.18 | 0.24 | 0.47 | 0.48 |
| frequency: per 1000 | 11.1 | 13.8 | 29.9 | 20.1 |
| (number) | (906) | (1118) | (2433) | (1634) |
| triplet | AUG | ACG | AAG | AGG |
| amino acid | M | T | K | R |
| fraction | 1.00 | 0.11 | 0.53 | 0.16 |
| frequency: per 1000 | 18.7 | 6.0 | 33.8 | 6.6 |
| (number) | (1517) | (491) | (2748) | (539) |
| triplet | GUU | GCU | GAU | GGU |
| amino acid | V | A | D | G |
| fraction | 0.42 | 0.45 | 0.58 | 0.44 |
| frequency: per 1000 | 26.9 | 28.9 | 35.7 | 25.5 |
| (number) | (2188) | (2351) | (2899) | (2075) |
| triplet | GUC | GCC | GAC | GGC |
| amino acid | V | A | D | G |
| fraction | 0.23 | 0.26 | 0.42 | 0.14 |
| frequency: per 1000 | 14.9 | 16.6 | 25.9 | 8.1 |
| (number) | (1210) | (1348) | (2103) | (655) |
| triplet | GUA | GCA | GAA | GGA |
| amino acid | V | A | E | G |
| fraction | 0.15 | 0.23 | 0.56 | 0.33 |
| frequency: per 1000 | 9.9 | 15.1 | 37.4 | 19.1 |
| (number) | (804) | (1228) | (3043) | (1550) |
| triplet | GUG | GCG | GAG | GGG |
| amino acid | V | A | E | G |
| fraction | 0.19 | 0.06 | 0.44 | 0.10 |
| frequency: per 1000 | 12.3 | 3.9 | 29.0 | 5.8 |
| (number) | (998) | (314) | (2360) | (468) |

### Exemplary Human CCR4 Binding Domains

As noted above, in some embodiments, the human CCR4 binding domains comprises an antigen-binding portion of an anti-CCR4 antibody, e g., an ScFv comprising the VH and VL regions of an anti-CCR4 antibody. An exemplary sequence of a codon-optimized cDNA encoding an anti-human CCR4 scFv including the V_{L} and V_{H} portions separated by three G₄S linkers is as follows, and can be used for both the monovalent and divalent toxins:

(SEQ ID NO: 6)
gacattgagttgactcaatctccatcttccttggctgtttctgctggtg
agaaggttactatgtcttgtaagtcttcccaatctattttgtactcttc
caaccaaaagaactacttggcttggtaccaacaaaagccaggtcaatct
ccaaagttgttgatttactgggcttctactagagagtctggtgttccag
acagattcactggttctggttctggtactgacttcactttgactatttc
ttccgttcaagctgaggacttggctgatactactgtcaccaatacttgt
cttcctacactttcggtggtggtactaagttggagattaagggtggtgg
tggttctggtggtggtggatctggtggtggtggttctcaagttcaattg
caacaatctggtccagagttggttagaccaggtgcttctgttagaattt
cttgtaaggcttctggttacactttcgcttcttactacattcaatggat
gaagcaaagaccaggtcaaggtttggagtggattggttggattaaccca
ggtaacgttaacactaagtacaacgagaagttcaagggtaaggctactt
tgactgctgacaagtcttccactaccgcttacatgcaattgtcttcctt
gacttctgaggactctgctgtttacttctgtgctagatccacttactac
agaccattggactactggggtcaaggtactaccgttactgtttcttcc

The above sequence codes for the following anti-human CCR4 scFv amino acid sequence including the VL and VH portions separated by three G4S linkers (linkers underlined):

(SEQ ID NO: 7)
DIELTQSPSSLAVSAGEKVTMSCKSSQSILYSSNQKNYLAWYQQKPGQSP
KLLIYWASTRESGVPDRFTGSGSGTDFTLTISSVQAEDLAVYYCHQYLSSY
TFGGGTKLEIKGGGGSGGGGSGGGGSQVQLQQSGPELVRPGASVRISCKA
SGYTFASYYIQWMKQRPGQGLEWIGWINPGNVNTKYNEKFKGKATLTA
DKSSTTAYMQLSSLTSEDSAVYFCARSTYYRPLDYWGQGTTVTVSS

An exemplary sequence of a codon-optimized DNA encoding an anti-human CCR4 scFv including the V_{L} and V_{H} portions separated by one G₄S linker is as follows, and can be used for the diabody toxins:

(SEQ ID NO: 8)
gacattgagttgactcaatctccatcttccttggctgtttctgctggtga
gaaggttactatgtcttgtaagtcttcccaatctattttgtactcttcca
accaaaagaactacttggcttggtaccaacaaaagccaggtcaatctcca
aagttgttgatttactgggcttctactagagagtctggtgttccagacag
attcactggttctggttctggtactgacttcactttgactatttcttccg
ttcaagctgaggacttggctgatactactgtcaccaatacttgtcttcct
acactttcggtggtggtactaagttggagattaagggtggtggtggatct
caagttcaattgcaacaatctggtccagagttggttagaccaggtgcttc
tgttagaatttcttgtaaggcttctggttacactttcgcttcttactaca
ttcaatggatgaagcaaagaccaggtcaaggtttggagtggattggttgg
attaacccaggtaacgttaacactaagtacaacgagaagttcaagggtaa
ggctactttgactgctgacaagtcttccactaccgcttacatgcaattgt
cttccttgacttctgaggactctgctgtttacttctgtgctagatccact
tactacagaccattggactactggggtcaaggtactaccgttactgtttc
ttcc

The above sequence codes for the following anti-human CCR4 scFv including the V_{L} and V_{H} portions separated by one G₄S linker (linker underlined):

(SEQ ID NO: 9)
DIELTQSPSSLAVSAGEKVTMSCKSSQSILYSSNQKNYLAWYQQKPGQSP
KLLIYWASTRESGVPDRFTGSGSGTDFTLTISSVQAEDLAVYYCHQYLSS
YTFGGGTKLEIKGGGGSQVQLQQSGPELVRPGASVRISCKASGYTFASYY
IQWMKQRPGQGLEWIGWINPGNVNTKYNEKFKGKATLTADKSSTTAYMQL
SSLTSEDSAVYFCARSTYYRPLDYWGQGTTVTVSS

Other linkers can be substituted for the linkers shown above, so long as they do not interfere with binding of the domain to the target CCR4.

Protein Production Methods

The methods for producing monovalent, bivalent, or single chain foldback diabody anti-human CCR4 immunotoxins described herein can be performed using protein production methods known in the art. For example, for scaled-up production, fermentation expression can be used.

Furthermore, although in a preferred embodiment the present methods use P. pastoris as a host organism, e.g., wild-type, X33, GS115 (his4), KM71, MC100-3, SMD1163, SMD1165, or SMD1168 strain, others can also be used. Other yeast, e.g., other methylotropic yeast, e.g., yeast of the genera Candida, Hansenula or Torulopsis, can also be used. Generally speaking, most P. Pastoris expression strains are derivatives of NRRL-Y 11430 (Northern Regional Research Laboratories, Peoria, Ill.). Other organisms can also be used.

Vectors suitable for use in the present methods are known in the art, and generally include a promoter, e.g., an AOX1, a constitutive P. Pastoris promoter derived from the P. Pastoris glyceraldehyde-3-phosphate dehydrogenase gene (GAP) promoter, typically followed immediately with a DNA sequence that encodes a secretion signal, e.g., the S. cerevisiae a factor prepro signal sequence, or the signal sequence derived from the P. Pastoris acid phosphatase gene (PHO1).

The vectors can also include one or more yeast selectable markers that can be used to identify and/or select those cells that contain the vector can be used. Such markers can include drug resistance markers and pathways for synthesis of essential cellular components, e.g., nutrients. Drug resistance markers that can be used in yeast include chloramphenicol, kanamycin, methotrexate, G418 (geneticin), Zeocin, and the like. Markers in synthesis pathways can be used with available yeast strains having auxotrophic mutations in the corresponding gene; examples include the pathways for synthesizing leucine (LEU2), tryptophan (TRP1 and TRP2), proline (PRO1), uracil (URA3, URA5, URA6), histidine (HIS3), lysine (LYS2), adenine (ADEJ or ADE2), and the like. Other yeast selectable markers include the ARR3 gene from S. cerevisiae, which confers arsenite resistance to yeast cells that are grown in the presence of arsenite (Bobrowicz et al., Yeast, 13:819-828 (1997); Wysocki et al, J-Biol. Chem. 272:30061-30066 (1997)). A number of suitable integration sites include those enumerated in U.S. Pat. No. 7,479,389 and include homologs to loci known for Saccharomyces cerevisiae and other yeast or fungi. Methods for integrating vectors into yeast are well known (See for example, U.S. Pat. No. 7,479,389, U.S. Pat. No. 7,514,253, U.S. Published Application No. 2009012400, and WO2009/085135). Examples of insertion sites include, but are not limited to, Pichia ADE genes; Pichia TRP (including TRP J through TRP2) genes; Pichia MCA genes; Pichia CYM genes; Pichia PEP genes; Pichia PRB genes; and Pichia LEU genes. The Pichia ADE1 and ARG4 genes have been described in Lin Cereghino et al, Gene 263:159-169 (2001) and U.S. Pat. No. 4,818,700, the HIS3 and TRP1 genes have been described in Cosano et al., Yeast 14:861-867 (1998), HIS4 has been described in GenBank Accession No. X5 180. See e.g., WO2011046855; Cregg, J. M. (2007) Methods in Molecular Biology: Pichia Protocols, Second Edition, Volume 389, Humana Press, Totowa, N.J.; Romanos et al., Yeast 8:423-488 (1992); Ilgen, et al., (2004) Chapter 7: Pichia Pastoris. In: Production of recombinant proteins: microbial and eukaryotic expression systems. Gellissen, G. (ed.) Wiley-VCH Verlag, Weinheim, Germany, pp. 143-162; Cereghino and Cregg, FEMS Microbiology Reviews 24:45-66 (2000); and Cregg, “The Pichia System”, available online at pichia.com/pichia_system.pdf. Exemplary vectors include pPIC3K, pPIC9K, pAO815 and the pPICZ vector series.

Purification

Methods known in the art can be used for nickel-based purification of the all three versions of the anti-human CCR4 immunotoxins. For example, although the present examples use a hexahistidine tag to facilitate purification, this may not be preferred for a pharmaceutical intended for in vivo use. Thus, other methods, including ammonium sulfate precipitation, reversed phase chromatography, hydrophobic interaction chromatography (HIC), size exclusion chromatography, ion exchange chromatography, affinity chromatography, metal binding, immunoaffinity chromatography, HPLC, or purification tags (e.g., as described above) may be used to directly capture the purified proteins. See, e.g., Deutscher, M. P. (1990) Guide to Protein Purification. In: Methods in Enzymology (J. N. Abelson and M. I. Simon, eds.) Academic Press, San Diego, Calif.; and The Recombinant Protein Handbook, Protein Amplification and Simple Purification, Amersham Biosciences, available online at 130.15.90.245/methods/hand-books%20and%20manuals/the%20recombinant%20protein%20handbook.pdf.

After purification, the protein can optionally be concentrated, e.g., by lyophilization or ultrafiltration.

Methods of Use

While Tregs function advantageously in development of transplantation tolerance and prevention of autoimmunity, their down regulation of immune responses may impede the body's ability to clear tumorigenic cell populations. Tumor progression induces proliferation of two T cell populations: those that target cancer cells; and those that down-regulate the targeting population, allowing the cancer to progress. The immune modulating cell populations are a major obstruction to treatments designed to activate and expand cells capable of targeting tumor cells. FOXP3^{hi}CD45RA⁻CD25^{hi}Tregs suppress immune responses to tumors, therefore, methods that target and deplete this cell population in vivo could prove to be useful in improving cancer immunotherapy. Tregs, along with Th2 (involved in allergic disease, e.g., asthma (Panina-Bordignon et al., J Clin Invest. 107(11): 1357-1364, (2001))), are known to express high levels of CCR4.

The anti-human CCR4 immunotoxins described herein can be used in the treatment or study of certain disorders, e.g., allergic diseases as well as cancer.

For example, this immunotoxin can be used to directly target tumor cells that express CCR4 on the surface, e.g., a leukemia or lymphoma such as adult T-cell leukemia/lymphoma (Ishida et al., Cancer Sci. 97(11): 1139-1146 (2006)); skin homing cutaneous T cell lymphoma (Ferenczi et al., J Invest Dermatol 119(6): 1405-1410 (2002)); or acute T-cell lymphoblastic leukemia (Yoshie et al., Blood 99(5): (2002)). Methods known in the art can be used to identify subjects who have cancers that express CCR4. In a preferred embodiment, the methods are used to treat subjects who have cutaneous T cell lymphoma. In some embodiments, the methods include administering one or more additional therapeutic agents, e.g., one or more of Vorinostat, Bexarotene and Romidepsin.

In another embodiment, the immunotoxins described herein can also be used as an immunotherapy to target and deplete FOXP3^{hi}CD45RA⁻CD25^{hi}Treg cells that express CCR4, which are known to suppress the immune response to cancer (Menetrier-Caux et al., Targ Oncol (2012)7:15-28), e.g., in solid tumors such as carcinoma, sarcoma, or melanoma. Generally, the methods include administering a therapeutically effective amount of the anti-human CCR4 immunotoxins as described herein, alone or in combination with another active agent, to a subject who is in need of, or who has been determined to be in need of, such treatment. In some embodiments, the methods also include administering one or more immunotherapies for cancer, e.g., one or more therapies that promote anti-cancer immunity, including administering one or more of: dendritic cells or peptides with adjuvant, immune checkpoint inhibitors, DNA-based vaccines, cytokines (e.g., IL-2), cyclophosphamide, agonists of OX40 (OX40; CD134), anti-interleukin-2R immunotoxins, and/or antibodies such as anti-CD137, anti-PD1, PDL1, or anti-CTLA-4; see, e.g., Krüger et al., Histol Histopathol. 2007 June; 22(6):687-96; Eggermont et al., Semin Oncol. 2010 October; 37(5):455-9; Klinke D J 2nd, Mol Cancer. 2010 Sep. 15; 9:242; Alexandrescu et al., J Immunother. 2010 July-August; 33(6):570-90; Moschella et al., Ann N Y Acad Sci. 2010 April; 1194:169-78; Ganesan and Bakhshi, Natl Med J India. 2010 January-February; 23(1):21-7; Golovina and Vonderheide, Cancer J. 2010 July-August; 16(4):342-7; Hodi et al., The New England journal of medicine 2010 363:711-723; Pentcheva-Hoang et al., Immunological Reviews 2009 229:67-87; Brahmer et al., Journal of Clinical Oncology 2010 28:3167-3175; Lynch et al., Journal of Clinical Oncology 2012 30(17):2046; Weber, Current Opinion in Oncology 2011 23:163-169; Weber, Seminars in Oncology 2010 37:430-439; Topalian et al., 2012. The New England Journal of Medicine 366:2443-2454; and Higano et al., Cancer 2009 115:3670-3679.

In some embodiments, the methods include administering a composition comprising tumor-pulsed dendritic cells, e.g., as described in WO2009/114547 and references cited therein. Additional examples of immunotherapies include virus-based anti-cancer vaccines (e.g., adenovirus), formulations of Toll-like Receptor or RIG-I-like receptor ligands, Adoptive T cell therapy or other cell types. In some embodiments the immunotherapy is selected from the group consisting of BiovaxID (an autologous vaccine containing tumor-specific idiotype proteins from individual patient's lymphoma cells conjugated to keyhole limpet hemocyanin (KLH)); Provenge sipuleucel-T (an FDA-approved example of the use of autologous dendritic cells); Yervoy (a mAb against CTLA-4 (CD152), approved in 2011 for metastatic melanoma); tremelimumab (formerly ticilimumab, an anti-CTLA-4 mAb); IMA901 (a vaccine containing 10 tumor-associated peptides (TUMAPs)), alone or in combination with Sutent (a small molecule VEGF receptor tyrosine kinase inhibitor); GV1001 (a peptide vaccine with the sequence of human telomerase reverse transcriptase (hTERT), from Kael-Gemvax); Lucanix belagenpumatecel-L (four NSCLC cell lines carrying antisense oligonucleotides against transforming growth factor beta 2 (TGFB2)); Stimuvax (a liposomal vaccine containing a synthetic 25-amino acid peptide sequence from mucin 1 (MUC1; CD227)); Allovectin velimogene aliplasmid (a DNA plasmid encoding major histocompatibility complex (MHC) class I B7 (HLA-B7) complexed with lipid); BMS-936558 (ONO-4538) (a human mAb against PD-1); BMS-936559 (formerly MDX-1105) (a human mAb against PD-L1); Zelboraf (vemurafenib, an oral small molecule inhibitor of the oncogenic BRAF V600E mutation); Votrient (pazopanib, a small molecule VEGF receptor tyrosine kinase inhibitor); ISF35 or Lucatumumab (HCD122) (mAbs against CD40); GVAX (an allogeneic cancer vaccine engineered to secrete granulocyte macrophage-colony stimulating factor (GM-CSF)). See, e.g., Flanagan, “Immune Springboard,” Biocentury, Jun. 18, 2012 A5-A10 (2012), available at biocentury.com. In some embodiments, the immunotherapy comprises administration of an agent that effects CTLA4 blockade (e.g., Ipilumumab BMS), PD1-blockade (e.g., BMS-936558, BMS; CT-011, Curetech; MK-3475, Merck), CD137 activation (e.g., BMS-663513, BMS), PD-L1 blockade (e.g., BMS-936559, BMS), CD40 activation (e.g., CP-870893, Pfizer) and autologous dendritic cells (e.g., Provenge).

In addition to Tregs, CCR4 is constitutively expressed on T helper type 2 (Th2) cells. Th2 cells are known to cause allergic inflammation in asthma (e.g., in the lungs in patients with allergic asthma) and other allergic diseases (Endo et al., Trends Immunol. 35(2): 69-78, (2014)); therefore, depleting Th2 cells is expected to be beneficial in treating allergic diseases associated with CCR4+ Th2 cells including asthma, rhinitis, food allergy, and eczema (Panina-Bordignon et al., J Clin Invest. 107(11): 1357-1364, (2001); (Yoshie and Matsushima, Intl Immunol. epub, Aug. 2, 2014); (Mikhak et al., J Allergy Clin Immunol 123:67, (2009); and (Schuh et al., FASEB J. 16:1313, (2002)). Therefore the present methods can include administering the CCR4 immunotoxins described herein to treat allergic diseases such as asthma, rhinitis, food allergy, and eczema via depleting CCR4+ Th2 cells.

An additional application of these proteins is use as a research tool, e.g., to study the role of Treg in immune regulation and transplant rejection. Experimental and clinical data demonstrated that Treg, characterized as FOXP3^{hi}CD45RA⁻CD25^{hi}, have significantly reduced suppression function in animal models and patients with autoimmune diseases such as rheumatoid arthritis, multiple sclerosis and type I diabetes (Viglietta et al., J Exp Med 199, 971 (2004); Lindley et al., Diabetes 54, 92 (2005); Ehrenstein et al., J Exp Med 200, 277 (2004); Sakaguchi et al., Cell 133, 775 (2008)). A reagent capable of depleting Treg in vivo could offer a useful tool for researchers studying autoimmune diseases in animal models.

Treg are also extensively studied in transplantation in an effort to understand the immunological mechanisms behind tolerance and rejection of allogeneic and xenogeneic organs. Increased levels of FOXP3^{hi}CD45RA⁻CD25^{hi}Treg have been detected in donor kidneys of tolerant recipients in experimental animal models and clinical patients (Miyajima et al., 2011). It is unclear, however, what role Treg play in the induction and maintenance of tolerance of these allografts. Efficient targeting and depletion of Treg in vivo may aid in determining the mechanisms of how Treg facilitate the initiation of and subsequently sustain tolerance to transplanted organs.

Thus the methods can include administering the immunotoxins or nucleic acids encoding the immunotoxins to an animal, e.g., an animal model of an autoimmune disease or of transplant rejection, and evaluating one or more symptoms or parameters of the disease in the animal.

Gene Therapy

The nucleic acids described herein can be incorporated into a gene construct to be used as a part of a gene therapy protocol. Expression constructs of such components can be administered in any effective carrier, e.g., any formulation or composition capable of effectively delivering the component gene to cells in vivo. Approaches include insertion of the gene in viral vectors, including recombinant retroviruses, adenovirus, adeno-associated virus, lentivirus, and herpes simplex virus-1, or recombinant bacterial or eukaryotic plasmids. Viral vectors transfect cells directly; plasmid DNA can be delivered naked or with the help of, for example, cationic liposomes (lipofectamine) or derivatized (e.g., antibody conjugated), polylysine conjugates, gramacidin S, artificial viral envelopes or other such intracellular carriers, as well as direct injection of the gene construct or CaPO₄ precipitation carried out in vivo.

A preferred approach for in vivo introduction of nucleic acid into a cell is by use of a viral vector containing nucleic acid, e.g., a cDNA. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells that have taken up viral vector nucleic acid.

Retrovirus vectors and adeno-associated virus vectors can be used as a recombinant gene delivery system for the transfer of exogenous genes in vivo, particularly into humans. These vectors provide efficient delivery of genes into cells, and the transferred nucleic acids are stably integrated into the chromosomal DNA of the host. The development of specialized cell lines (termed “packaging cells”) which produce only replication-defective retroviruses has increased the utility of retroviruses for gene therapy, and defective retroviruses are characterized for use in gene transfer for gene therapy purposes (for a review see Miller, Blood 76:271 (1990)). A replication defective retrovirus can be packaged into virions, which can be used to infect a target cell through the use of a helper virus by standard techniques. Protocols for producing recombinant retroviruses and for infecting cells in vitro or in vivo with such viruses can be found in Ausubel, et al., eds., Current Protocols in Molecular Biology, Greene Publishing Associates, (1989), Sections 9.10-9.14, and other standard laboratory manuals. Examples of suitable retroviruses include pLJ, pZIP, pWE and pEM which are known to those skilled in the art. Examples of suitable packaging virus lines for preparing both ecotropic and amphotropic retroviral systems include psi-Crip, psi-Cre, psi-2 and psi-Am. Retroviruses have been used to introduce a variety of genes into many different cell types, including epithelial cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Another viral gene delivery system useful in the present methods utilizes adenovirus-derived vectors. The genome of an adenovirus can be manipulated, such that it encodes and expresses a gene product of interest but is inactivated in terms of its ability to replicate in a normal lytic viral life cycle. See, for example, Berkner et al., BioTechniques 6:616 (1988); Rosenfeld et al., Science 252:431-434 (1991); and Rosenfeld et al., Cell 68:143-155 (1992). Suitable adenoviral vectors derived from the adenovirus strain Ad type 5 dl324 or other strains of adenovirus (e.g., Ad2, Ad3, or Ad7 etc.) are known to those skilled in the art. Recombinant adenoviruses can be advantageous in certain circumstances, in that they are not capable of infecting non-dividing cells and can be used to infect a wide variety of cell types, including epithelial cells (Rosenfeld et al., (1992) supra). Furthermore, the virus particle is relatively stable and amenable to purification and concentration, and as above, can be modified so as to affect the spectrum of infectivity. Additionally, introduced adenoviral DNA (and foreign DNA contained therein) is not integrated into the genome of a host cell but remains episomal, thereby avoiding potential problems that can occur as a result of insertional mutagenesis in situ, where introduced DNA becomes integrated into the host genome (e.g., retroviral DNA). Moreover, the carrying capacity of the adenoviral genome for foreign DNA is large (up to 8 kilobases) relative to other gene delivery vectors (Berkner et al., supra; Haj-Ahmand and Graham, J. Virol. 57:267 (1986).

Yet another viral vector system useful for delivery of nucleic acids is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129 (1992). It is also one of the few viruses that may integrate its DNA into non-dividing cells, and exhibits a high frequency of stable integration (see for example Flotte et al., Am. J. Respir. Cell. Mol. Biol. 7:349-356 (1992); Samulski et al., J. Virol. 63:3822-3828 (1989); and McLaughlin et al., J. Virol. 62:1963-1973 (1989). Vectors containing as little as 300 base pairs of AAV can be packaged and can integrate. Space for exogenous DNA is limited to about 4.5 kb. An AAV vector such as that described in Tratschin et al., Mol. Cell. Biol. 5:3251-3260 (1985) can be used to introduce DNA into cells. A variety of nucleic acids have been introduced into different cell types using AAV vectors (see for example Hermonat et al., Proc. Natl. Acad. Sci. USA 81:6466-6470 (1984); Tratschin et al., Mol. Cell. Biol. 4:2072-2081 (1985); Wondisford et al., Mol. Endocrinol. 2:32-39 (1988); Tratschin et al., J. Virol. 51:611-619 (1984); and Flotte et al., J. Biol. Chem. 268:3781-3790 (1993).

In addition to viral transfer methods, such as those illustrated above, non-viral methods can also be employed to cause expression of a nucleic acid described herein in the tissue of a subject, e.g., in a tumor tissue. Typically non-viral methods of gene transfer rely on the normal mechanisms used by mammalian cells for the uptake and intracellular transport of macromolecules. In some embodiments, non-viral gene delivery systems can rely on endocytic pathways for the uptake of the subject gene by the targeted cell. Exemplary gene delivery systems of this type include liposomal derived systems, poly-lysine conjugates, and artificial viral envelopes. Other embodiments include plasmid injection systems such as are described in Meuli et al., J. Invest. Dermatol. 116(1):131-135 (2001); Cohen et al., Gene Ther. 7(22):1896-905 (2000); or Tam et al., Gene Ther. 7(21):1867-74 (2000).

In clinical settings, the gene delivery systems for the therapeutic gene can be introduced into a subject by any of a number of methods, each of which is known in the art. For instance, a pharmaceutical preparation of the gene delivery system can be introduced systemically, e.g., by intravenous injection, and specific transduction of the protein in the target cells will occur predominantly from specificity of transfection, provided by the gene delivery vehicle, cell-type or tissue-type expression due to the transcriptional regulatory sequences controlling expression of the receptor gene, or a combination thereof. In other embodiments, initial delivery of the recombinant gene is more limited, with introduction into the subject being quite localized. For example, the gene delivery vehicle can be introduced by catheter (see U.S. Pat. No. 5,328,470) or by stereotactic injection (e.g., Chen et al., PNAS USA 91: 3054-3057 (1994)).

The pharmaceutical preparation of the gene therapy construct can consist essentially of the gene delivery system in an acceptable diluent, or can comprise a slow release matrix in which the gene delivery vehicle is embedded.

### EXAMPLES

The invention is further described in the following examples, which do not limit the scope of the invention described in the claims.

### Example 1. Anti-Human CCR4 Immunotoxins

Example 1 describes the generation and testing of anti-human CCR4 immunotoxins.

The truncated diphtheria toxin DT390 has been used to build recombinant immunotoxins (Woo et al., Protein Expr. Purif. 25, 270-282 (2002); Kim et al., Protein Eng. Des. Sel. 20, 425-432 (2007); Wang et al., Bioconjug Chem. 22, 2014-2020 (2011); see also WO2001087982A2). DT390 lacks the cell-surface binding domain and consists of the catalytic and translocation domains of the diphtheria toxin. In the present study a mouse monoclonal anti-human CCR4 scFv protein was linked to DT390 through genetic engineering yielding exemplary anti-human CCR4 immunotoxins. The ability of these reagents to deplete target cells was assessed using in vitro assays that monitored the inhibition of protein synthesis and cell proliferation. Binding specificity and affinity to the target cells was analyzed by flow cytometry.

Materials and Methods

The following materials and methods were used in Example 1 set forth below.

Antibodies and Cell Line.

Human CCR4⁺ acute lymphoblastic leukemia cell line CCRF-CEM was purchased from ATCC (cat# CCL-119); non-CCR4 expressing tumor cell lines: MV3, M14 and MD-MBA-231 were generously provided by Dr. Soldano Ferrone (Massachusetts General Hospital). Human/rat CCR4 fluorescein mAb (clone 205410, cat# FAB1567F) and mouse IgG2B fluorescein isotype control (clone 133303, cat# IC0041F) were purchased from R&D Systems. PE-anti-human 194 (CCR4) mAb (Clone# L291H4), Alexa Fluor® 647 anti-human Foxp3 mAb (clone#150D, cat#320014) and Alex Fluor 647 Mouse IgG1 κ (clone# MOPC-21, cat#400136) were purchased from Biolegend. BiscFv(1567)-Human Fc was produced using yeast Pichia Pastoris expression system in our lab.

Plasmid Construction.

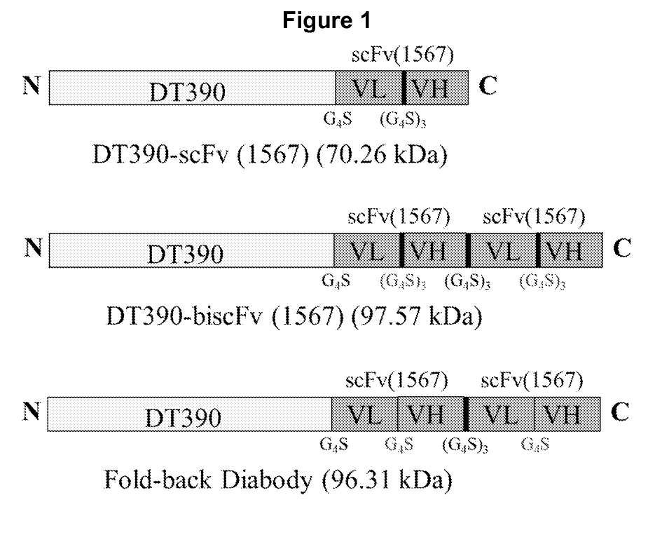

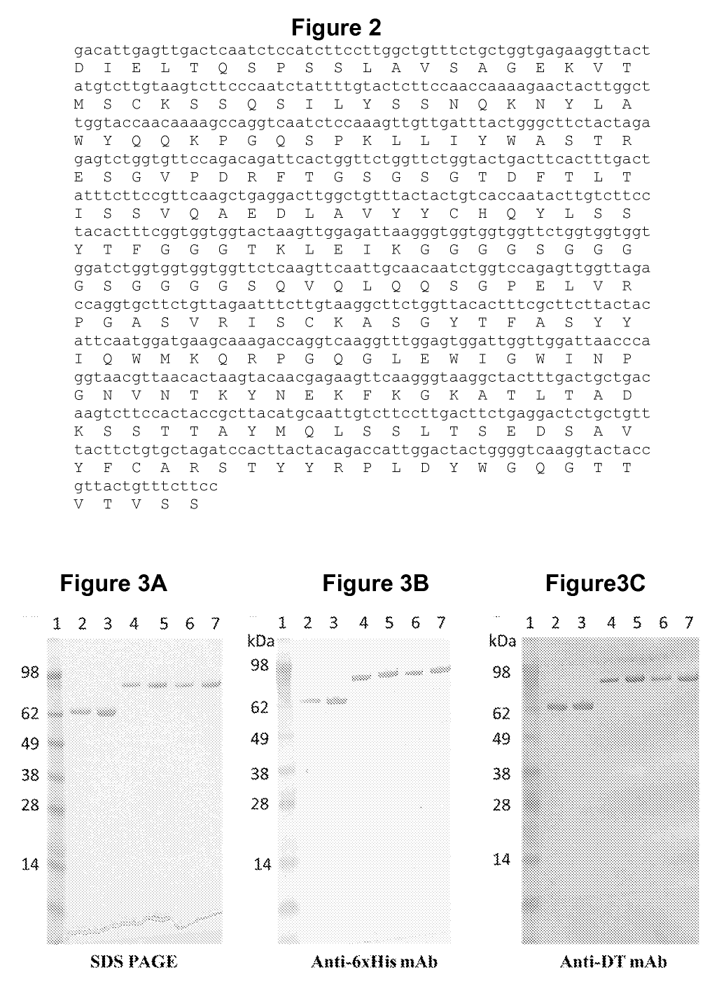

As shown in FIG. 1, anti-human CCR4 immunotoxins were built to contain two moieties using the codon-optimized nucleotide sequences; the first is DT390 (Woo et al., Protein Expr. Purif. 25, 270-282 (2002)) and the second is anti-human CCR4 scFv. A strategy previously employed to construct A-dm-DT390biscFv (2-6-15) (Wang et al., Bioconjug Chem. 22, 2014-2020 (2011)) was applied to build these anti-human CCR4 immunotoxins. The biscFv (2-6-15) moiety was replaced with the codon-optimized monovalent or bivalent anti-human CCR4 (FIG. 2). A linker made up of three tandem chains each containing four glycine residues and a serine (G₄S)₃ was used to connect one or two anti-human CCR4 proteins for building the monovalent or bivalent CCR4 immunotoxins. Six histidines (6×His tag) were added to the C-terminus of each construct to facilitate later purification. To construct the monovalent DT390-CCR4 immunotoxin, the codon-optimized anti-human CCR4 was synthesized by GenScript (Piscataway, N.J.) and cloned into pwPICZalpha-DT390 (Wang et al., Bioconjug Chem. 22, 2014-2020 (2011)) between NcoI and EcoRI sites yielding the final construct DT390-CCR4 in pwPICZalpha. To construct an exemplary bivalent DT390-CCR4 immunotoxin (also referred to as DT390-BiscFv(1567)), the first scFv(1567) was amplified using PCR primers CCR4-Nco carrying XhoI and NcoI sites+CCR4-Bam1 carrying BamHI and EcoRI sites then cloned into pwPICZalpha between XhoI and EcoRI sites for sequencing confirmation. The insert was subsequently cut out with NcoI+BamHI as insert I. The second scFv (1567) was PCR amplified using CCR4-Bam2 carrying XhoI and BamHI sites+CCR4-Eco carrying an EcoRI site then cloned into pwPICZalpha between XhoI and EcoRI sites for sequencing confirmation. The insert was then cut out with BamHI+EcoRI as insert II. The insert I carrying NcoI and BamHI sites+insert II carrying BamHI and EcoRI sites (NcoI-CCR4-BamHI/BamHI-CCR4-EcoRI) were together cloned into pwPICZalpha-DT390 between NcoI and EcoRI yielding the final construct DT390-BiCCR4 in pwPICZalpha. To construct the single-chain foldback diabody isoform, it is required to build a modified scFv (1567) with a short linker (one G₄S) between V_{L} and V_{H} portions. The V_{L} portion was amplified using PCR primers CCR4-Nco+Bam-CCR4 carrying BamHI site and digested using BamHI. The V_{H} portion was amplified using PCR primers BgL-CCR4 carrying BglII site+CCR4-Bam1 and digested using Bgl II. The BamHI-digested VL portion and Bgl II-digested VH portion were ligated together for 4 h at room temperature as template to construct the single-chain fold-back diabody isoform following the constructing procedure for DT390-BiCCR4. All PCR primers that were used are listed in Table 2.

TABLE 2
PCR primers used in this study

| | | SEQ ID |
|---|---|---|
| PRIMER | SEQUENCE | NO: |
| CCR4-Nco | 5′ CCG CTC GAG CCA TGG GGT GGT GGT GGT | 10 |
| | TCT GAC ATT GAG TTG ACT CAA TCT CCA 3′ | |
| CCR4-Bam1 | 5′ CCG GAA TTC CGC CGC GGA TCC ACC ACC | 11 |
| | ACC AGA ACC ACC ACC ACC GGA AGA AAC | |
| | AGT AAC GGT AGT 3′ | |
| CCR4-Bam2 | 5′ CCG CTC GAG GGA TCC GGT GGT GGT GGT | 12 |
| | TCT GAC ATT GAG TTG ACT CAA TCT CCA 3′ | |
| CCR4-Eco | 5′ CCG GAA TTC TTA GTG GTG GTG GTG GTG | 13 |
| | GTG GGA AGA AAC AGT AAC GGT AGT 3′ | |
| Bam-CCR4 | 5′ CGC GGA TCC ACC ACC ACC CTT AAT CTC | 14 |
| | CAA CTT AGT ACC AC 3′ | |
| BgL-CCR4 | 5′ GGA AGA TCT CAA GTT CAA TTG CAA CAA | 15 |
| | TCT GG 3′ | |

Protein expression and purification in Pichia Pastoris were performed as previously described (Wang et al., (2011) supra; Peraino et al., Protein Expr Purif. 82, 270-278 (2012)). Western blot analysis and blocking analysis by flow cytometry were performed as previously described (Peraino et al., Protein Expr Purif. 82, 270-278 (2012)) using a human CCR4+ acute lymphoblastic leukemia cell line CCRF-CEM (ATCC CCL-119).

Protein synthesis inhibition and cell proliferation inhibition were performed as described previously (Peraino et al 2014). Using BiscFv (1567)-human Fc anti-human CCR4 antibody as inhibitor to block the protein synthesis inhibition and cell proliferation inhibition of the anti-human CCR4 immunotoxins was also performed as described by Peraino et al 2014. Isolation of human PBMC, in vitro binding and depletion analysis of the anti-human CCR4 immunotoxins to human CCR4 on PBMC using flow cytometry was performed as previously described (Peraino et al., 2013b, 2014).

HPLC Analysis.

Anti-human CCR4 immunotoxins were analyzed with Shimadzu HPLC system using Superdex 200 size-exclusion column, 10/300 GL (GE healthcare, Cat#: 17-5175-01). The sample volume was 100 μl using 100 μl loop. The flow rate was 0.35 ml/min. The running time was 120 min and the running buffer was 90 mM Na2SO4, 10 mM Na3PO4, pH 8.0, 1 mM EDTA.

In Vivo Efficacy Study.

A breeding pair of NSG mice were purchased from Jackson laboratories and bred in our rodent barrier facilities for use in this study. All animal care procedures and experiments were approved by the Institutional Animal Care and Use Committee (IACUC) of Massachusetts General Hospital (MGH).

The NSG mice were divided into one control group and three experimental groups: 1) C21 immunotoxin control group (a non-related diphtheria toxin-based immunotoxin as negative control) (n=7); 2) monovalent anti-human CCR4 immunotoxin group (n=7); 3) bivalent anti-human CCR4 immunotoxin group (n=8); 4) single-chain foldback diabody anti-human CCR4 immunotoxin group (n=7). All animals were IV injected with 10 million of human CCR4+ acute lymphoblastic leukemia cells (CCRF-CEM) via the tail vein. The immunotoxin was IP injected from day 0 on at 50 μg/kg, BID for 4 consecutive days as one course, two course total, and 3-day break between the two courses. The injected animals were observed daily for signs and symptoms of illness and scored weekly based on the parameters as previously reported by our lab (Peraino et al., 2013b): respiratory effort (0-3), weight loss/gain (0-2), fur integrity (0-3), provoked (0-3) and non-provoked activity (0-1), posture (0-3), abdominal distention (0-3), abdominal palpation (0-3) and body condition score (0-3). The highest score in each category represents the worst possible condition for that parameter. The highest possible score on the scoring system is a 24. The animals were humanely euthanized after a score of 12 or higher or when an animal lost more than 15% of its pre-injection body weight. However this score system was actually almost not applied as the CCR4+ ALL is very aggressive to result in the animal death or euthanized within very short period of time. It was less than or about one day from completely normal to dead. To assess whether the immunotoxin alone is toxic to the experimental animals, mice (n=2) injected with the bivalent or single-chain fold-back diabody immunotoxin only (without tumor cells) were also included as controls.

Statistical Analysis.

All P values were calculated using two-way ANOVA or Log-rank (Mantel-Cox) Test of Prism. P<0.05 was considered as significant. EC50 was determined using nonlinear regression (curve fit) of Prism.

### Example 1.1. Expression and Purification of Anti-Human CCR4 Immunotoxins

As shown in FIG. 1, monovalent, bivalent and single-chain foldback diabody anti-human CCR4 toxins were constructed so as to find the best isoform for in vivo depleting human CCR4+ Tregs. The codon-optimized anti-human CCR4 scFv DNA (FIG. 2) was cloned into the C-terminus of the DT390-containing yeast Pichia Pastoris expression vector pwPICZalpha-DT390 between the NcoI and EcoRI sites (Wang et al., 2011). A 6×his tag was added to the C-terminus of each immunotoxin construct to facilitate the downstream purification. The DT390 domain was genetically linked to the anti-human CCR4 domain by a linker containing four glycine residues and a serine residue (G4S). For the monovalent and bivalent version, the VL and VH were linked together by three tandem G4S linker (G4S)₃ to generate the anti-human CCR4 immunotoxins. For the single-chain foldback diabody isoform, the V_{L} and V_{H} were linked together by one G₄S linker. The two anti-human CCR4 which make up the bivalent and the single-chain foldback diabody isoforms were also joined by three tandem G₄S linkers (G₄S)₃.

Amino acid and nucleic acid sequences for the monovalent, bivalent, and version single-chain foldback diabody isoforms were as follows.

DT390-scFv(1567)-6xHis DNA sequnece:
GCTGGTGCTGACGACGTCGTCGACTCCTCCAAGTCCTTCGTCATGGAGAA
CTTCGCTTCCTACCACGGGACCAAGCCAGGTTACGTCGACTCCATCCAGA
AGGGTATCCAGAAGCCAAAGTCCGGCACCCAAGGTAACTACGACGACGAC
TGGAAGGGGTTCTACTCCACCGACAACAAGTACGACGCTGCGGGATACTC
TGTAGATAATGAAAACCCGCTCTCTGGAAAAGCTGGAGGCGTGGTCAAGG
TCACCTACCCAGGTCTGACTAAGGTCTTGGCTTTGAAGGTCGACAACGCT
GAGACCATCAAGAAGGAGTTGGGTTTGTCCTTGACTGAGCCATTGATGGA
GCAAGTCGGTACCGAAGAGTTCATCAAGAGATTCGGTGACGGTGCTTCCA
GAGTCGTCTTGTCCTTGCCATTCGCTGAGGGTTCTTCTAGCGTTGAATAT
ATTAATAACTGGGAACAGGCTAAGGCTTTGTCTGTTGAATTGGAGATTAA
CTTCGAAACCAGAGGTAAGAGAGGTCAAGATGCGATGTATGAGTATATGG
CTCAAGCCTGTGCTGGTAACAGAGTCAGACGTTCTGTTGGTTCCTCTTTG
TCCTGTATCAACCTAGACTGGGACGTCATCAGAGACAAGACTAAGACCAA
GATCGAGTCTTTGAAAGAGCATGGCCCAATCAAGAACAAGATGTCCGAAT
CCCCCGCTAAGACCGTCTCCGAGGAAAAGGCCAAGCAATACCTAGAAGAG
TTCCACCAAACCGCCTTGGAGCATCCTGAATTGTCAGAACTTAAAACCGT
TACTGGGACCAATCCTGTATTCGCTGGGGCTAACTATGCGGCGTGGGCAG
TAAACGTTGCGCAAGTTATCGATAGCGAAACAGCTGATAATTTGGAAAAG
ACAACTGCTGCTCTTTCGATACTTCCTGGTATCGGTAGCGTAATGGGCAT
TGCAGACGGTGCCGTTCACCACAATACAGAAGAGATAGTGGCACAATCCA
TCGCTTTGTCCTCTTTGATGGTTGCTCAAGCTATCCCATTGGTCGGTGAG
TTGGTTGACATCGGTTTCGCTGCCTACAACTTCGTCGAGTCCATCATCAA
CTTGTTCCAAGTCGTCCACAACTCCTACAACCGTCCGGCTTACTCCCCAG
GTCACAAGACCCAACCATTCTTGCCATGGGGTGGTGGTGGTTCTGACATT
GAGTTGACTCAATCTCCATCTTCCTTGGCTGTTTCTGCTGGTGAGAAGGT
TACTATGTCTTGTAAGTCTTCCCAATCTATTTTGTACTCTTCCAACCAAA
AGAACTACTTGGCTTGGTACCAACAAAAGCCAGGTCAATCTCCAAAGTTG
TTGATTTACTGGGCTTCTACTAGAGAGTCTGGTGTTCCAGACAGATTCAC
TGGTTCTGGTTCTGGTACTGACTTCACTTTGACTATTTCTTCCGTTCAAG
CTGAGGACTTGGCTGTTTACTACTGTCACCAATACTTGTCTTCCTACACT
TTCGGTGGTGGTACTAAGTTGGAGATTAAGGGTGGTGGTGGTTCTGGTGG
TGGTGGATCTGGTGGTGGTGGTTCTCAAGTTCAATTGCAACAATCTGGTC
CAGAGTTGGTTAGACCAGGTGCTTCTGTTAGAATTTCTTGTAAGGCTTCT
GGTTACACTTTCGCTTCTTACTACATTCAATGGATGAAGCAAAGACCAGG
TCAAGGTTTGGAGTGGATTGGTTGGATTAACCCAGGTAACGTTAACACTA
AGTACAACGAGAAGTTCAAGGGTAAGGCTACTTTGACTGCTGACAAGTCT
TCCACTACCGCTTACATGCAATTGTCTTCCTTGACTTCTGAGGACTCTGC
TGTTTACTTCTGTGCTAGATCcACTTACTACAGACCATTGGACTACTGGG
GTCAAGGTACTACCGTTACTGTTTCTTCCCACCACCACCACCACCAC
DT390-scFv(1567)-6xHis amino acid sequence:
AGADDVVDSSKSFVMENFASYHGTKPGYVDSIQKGIQKPKSGTQGNYDDD
WKGFYSTDNKYDAAGYSVDNENPLSGKAGGVVKVTYPGLTKVLALKVDNA
ETIKKELGLSLTEPLMEQVGTEEFIKRFGDGASRVVLSLPFAEGSSSVEY
INNWEQAKALSVELEINFETRGKRGQDAMYEYMAQACAGNRVRRSVGSSL
SCINLDWDVIRDKTKTKIESLKEHGPIKNKMSESPAKTVSEEKAKQYLEE
FHQTALEHPELSELKTVTGTNPVFAGANYAAWAVNVAQVIDSETADNLEK
TTAALSILPGIGSVMGIADGAVHHNTEEIVAQSIALSSLMVAQAIPLVGE
LVDIGFAAYNFVESIINLFQVVHNSYNRPAYSPGHKTQPFLPWGGGGSDI
ELTQSPSSLAVSAGEKVTMSCKSSQSILYSSNQKNYLAWYQQKPGQSPKL
LIYWASTRESGVPDRFTGSGSGTDFTLTISSVQAEDLAVYYCHQYLSSYT
FGGGTKLEIKGGGGSGGGGSGGGGSQVQLQQSGPELVRPGASVRISCKAS
GYTFASYYIQWMKQRPGQGLEWIGWINPGNVNTKYNEKFKGKATLTADKS
STTAYMQLSSLTSEDSAVYFCARSTYYRPLDYWGQGTTVTVSSHHHHHH
DT390-biscFv(1567) DNA sequence:
GCTGGTGCTGACGACGTCGTCGACTCCTCCAAGTCCTTCGTCATGGAGAA
CTTCGCTTCCTACCACGGGACCAAGCCAGGTTACGTCGACTCCATCCAGA
AGGGTATCCAGAAGCCAAAGTCCGGCACCCAAGGTAACTACGACGACGAC
TGGAAGGGGTTCTACTCCACCGACAACAAGTACGACGCTGCGGGATACTC
TGTAGATAATGAAAACCCGCTCTCTGGAAAAGCTGGAGGCGTGGTCAAGG
TCACCTACCCAGGTCTGACTAAGGTCTTGGCTTTGAAGGTCGACAACGCT
GAGACCATCAAGAAGGAGTTGGGTTTGTCCTTGACTGAGCCATTGATGGA
GCAAGTCGGTACCGAAGAGTTCATCAAGAGATTCGGTGACGGTGCTTCCA
GAGTCGTCTTGTCCTTGCCATTCGCTGAGGGTTCTTCTAGCGTTGAATAT
ATTAATAACTGGGAACAGGCTAAGGCTTTGTCTGTTGAATTGGAGATTAA
CTTCGAAACCAGAGGTAAGAGAGGTCAAGATGCGATGTATGAGTATATGG
CTCAAGCCTGTGCTGGTAACAGAGTCAGACGTTCTGTTGGTTCCTCTTTG
TCCTGTATCAACCTAGACTGGGACGTCATCAGAGACAAGACTAAGACCAA
GATCGAGTCTTTGAAAGAGCATGGCCCAATCAAGAACAAGATGTCCGAAT
CCCCCGCTAAGACCGTCTCCGAGGAAAAGGCCAAGCAATACCTAGAAGAG
TTCCACCAAACCGCCTTGGAGCATCCTGAATTGTCAGAACTTAAAACCGT
TACTGGGACCAATCCTGTATTCGCTGGGGCTAACTATGCGGCGTGGGCAG
TAAACGTTGCGCAAGTTATCGATAGCGAAACAGCTGATAATTTGGAAAAG
ACAACTGCTGCTCTTTCGATACTTCCTGGTATCGGTAGCGTAATGGGCAT
TGCAGACGGTGCCGTTCACCACAATACAGAAGAGATAGTGGCACAATCCA
TCGCTTTGTCCTCTTTGATGGTTGCTCAAGCTATCCCATTGGTCGGTGAG
TTGGTTGACATCGGTTTCGCTGCCTACAACTTCGTCGAGTCCATCATCAA
CTTGTTCCAAGTCGTCCACAACTCCTACAACCGTCCGGCTTACTCCCCAG
GTCACAAGACCCAACCATTCTTGCCATGGGGTGGTGGTGGTTCTGACATT
GAGTTGACTCAATCTCCATCTTCCTTGGCTGTTTCTGCTGGTGAGAAGGT
TACTATGTCTTGTAAGTCTTCCCAATCTATTTTGTACTCTTCCAACCAAA
AGAACTACTTGGCTTGGTACCAACAAAAGCCAGGTCAATCTCCAAAGTTG
TTGATTTACTGGGCTTCTACTAGAGAGTCTGGTGTTCCAGACAGATTCAC
TGGTTCTGGTTCTGGTACTGACTTCACTTTGACTATTTCTTCCGTTCAAG
CTGAGGACTTGGCTGTTTACTACTGTCACCAATACTTGTCTTCCTACACT
TTCGGTGGTGGTACTAAGTTGGAGATTAAGGGTGGTGGTGGTTCTGGTGG
TGGTGGATCTGGTGGTGGTGGTTCTCAAGTTCAATTGCAACAATCTGGTC
CAGAGTTGGTTAGACCAGGTGCTTCTGTTAGAATTTCTTGTAAGGCTTCT
GGTTACACTTTCGCTTCTTACTACATTCAATGGATGAAGCAAAGACCAGG
TCAAGGTTTGGAGTGGATTGGTTGGATTAACCCAGGTAACGTTAACACTA
AGTACAACGAGAAGTTCAAGGGTAAGGCTACTTTGACTGCTGACAAGTCT
TCCACTACCGCTTACATGCAATTGTCTTCCTTGACTTCTGAGGACTCTGC
TGTTTACTTCTGTGCTAGATCcACTTACTACAGACCATTGGACTACTGGG
GTCAAGGTACTACCGTTACTGTTTCTTCCGGTGGTGGTGGTTCTGGTGGT
GGTGGATCCGGTGGTGGTGGTTCTGACATTGAGTTGACTCAATCTCCATC
TTCCTTGGCTGTTTCTGCTGGTGAGAAGGTTACTATGTCTTGTAAGTCTT
CCCAATCTATTTTGTACTCTTCCAACCAAAAGAACTACTTGGCTTGGTAC
CAACAAAAGCCAGGTCAATCTCCAAAGTTGTTGATTTACTGGGCTTCTAC
TAGAGAGTCTGGTGTTCCAGACAGATTCACTGGTTCTGGTTCTGGTACTG
ACTTCACTTTGACTATTTCTTCCGTTCAAGCTGAGGACTTGGCTGTTTAC
TACTGTCACCAATACTTGTCTTCCTACACTTTCGGTGGTGGTACTAAGTT
GGAGATTAAGGGTGGTGGTGGTTCTGGTGGTGGTGGATCTGGTGGTGGTG
GTTCTCAAGTTCAATTGCAACAATCTGGTCCAGAGTTGGTTAGACCAGGT
GCTTCTGTTAGAATTTCTTGTAAGGCTTCTGGTTACACTTTCGCTTCTTA
CTACATTCAATGGATGAAGCAAAGACCAGGTCAAGGTTTGGAGTGGATTG
GTTGGATTAACCCAGGTAACGTTAACACTAAGTACAACGAGAAGTTCAAG
GGTAAGGCTACTTTGACTGCTGACAAGTCTTCCACTACCGCTTACATGCA
ATTGTCTTCCTTGACTTCTGAGGACTCTGCTGTTTACTTCTGTGCTAGAT
CcACTTACTACAGACCATTGGACTACTGGGGTCAAGGTACTACCGTTACT
GTTTCTTCCCACCACCACCACCACCAC
DT390-biscFv(1567)-6xHis, amino acid sequence:
AGADDVVDSSKSFVMENFASYHGTKPGYVDSIQKGIQKPKSGTQGNYDDD
WKGFYSTDNKYDAAGYSVDNENPLSGKAGGVVKVTYPGLTKVLALKVDNA
ETIKKELGLSLTEPLMEQVGTEEFIKRFGDGASRVVLSLPFAEGSSSVEY
INNWEQAKALSVELEINFETRGKRGQDAMYEYMAQACAGNRVRRSVGSSL
SCINLDWDVIRDKTKTKIESLKEHGPIKNKMSESPAKTVSEEKAKQYLEE
FHQTALEHPELSELKTVTGTNPVFAGANYAAWAVNVAQVIDSETADNLEK
TTAALSILPGIGSVMGIADGAVHHNTEEIVAQSIALSSLMVAQAIPLVGE
LVDIGFAAYNFVESIINLFQVVHNSYNRPAYSPGHKTQPFLPWGGGGSDI
ELTQSPSSLAVSAGEKVTMSCKSSQSILYSSNQKNYLAWYQQKPGQSPKL
LIYWASTRESGVPDRFTGSGSGTDFTLTISSVQAEDLAVYYCHQYLSSYT
FGGGTKLEIKGGGGSGGGGSGGGGSQVQLQQSGPELVRPGASVRISCKAS
GYTFASYYIQWMKQRPGQGLEWIGWINPGNVNTKYNEKFKGKATLTADKS
STTAYMQLSSLTSEDSAVYFCARSTYYRPLDYWGQGTTVTVSSGGGGSGG
GGSGGGGSDIELTQSPSSLAVSAGEKVTMSCKSSQSILYSSNQKNYLAWY
QQKPGQSPKLLIYWASTRESGVPDRFTGSGSGTDFTLTISSVQAEDLAVY
YCHQYLSSYTFGGGTKLEIKGGGGSGGGGSGGGGSQVQLQQSGPELVRPG
ASVRISCKASGYTFASYYIQWMKQRPGQGLEWIGWINPGNVNTKYNEKFK
GKATLTADKSSTTAYMQLSSLTSEDSAVYFCARSTYYRPLDYWGQGTTVT
VSSHHHHHH
DT390-single-chain foldback diabody anti-human
CCR4 immunotoxin-6xHis, DNA seqence:
GCTGGTGCTGACGACGTCGTCGACTCCTCCAAGTCCTTCGTCATGGAGAA
CTTCGCTTCCTACCACGGGACCAAGCCAGGTTACGTCGACTCCATCCAGA
AGGGTATCCAGAAGCCAAAGTCCGGCACCCAAGGTAACTACGACGACGAC
TGGAAGGGGTTCTACTCCACCGACAACAAGTACGACGCTGCGGGATACTC
TGTAGATAATGAAAACCCGCTCTCTGGAAAAGCTGGAGGCGTGGTCAAGG
TCACCTACCCAGGTCTGACTAAGGTCTTGGCTTTGAAGGTCGACAACGCT
GAGACCATCAAGAAGGAGTTGGGTTTGTCCTTGACTGAGCCATTGATGGA
GCAAGTCGGTACCGAAGAGTTCATCAAGAGATTCGGTGACGGTGCTTCCA
GAGTCGTCTTGTCCTTGCCATTCGCTGAGGGTTCTTCTAGCGTTGAATAT
ATTAATAACTGGGAACAGGCTAAGGCTTTGTCTGTTGAATTGGAGATTAA
CTTCGAAACCAGAGGTAAGAGAGGTCAAGATGCGATGTATGAGTATATGG
CTCAAGCCTGTGCTGGTAACAGAGTCAGACGTTCTGTTGGTTCCTCTTTG
TCCTGTATCAACCTAGACTGGGACGTCATCAGAGACAAGACTAAGACCAA
GATCGAGTCTTTGAAAGAGCATGGCCCAATCAAGAACAAGATGTCCGAAT
CCCCCGCTAAGACCGTCTCCGAGGAAAAGGCCAAGCAATACCTAGAAGAG
TTCCACCAAACCGCCTTGGAGCATCCTGAATTGTCAGAACTTAAAACCGT
TACTGGGACCAATCCTGTATTCGCTGGGGCTAACTATGCGGCGTGGGCAG
TAAACGTTGCGCAAGTTATCGATAGCGAAACAGCTGATAATTTGGAAAAG
ACAACTGCTGCTCTTTCGATACTTCCTGGTATCGGTAGCGTAATGGGCAT
TGCAGACGGTGCCGTTCACCACAATACAGAAGAGATAGTGGCACAATCCA
TCGCTTTGTCCTCTTTGATGGTTGCTCAAGCTATCCCATTGGTCGGTGAG
TTGGTTGACATCGGTTTCGCTGCCTACAACTTCGTCGAGTCCATCATCAA
CTTGTTCCAAGTCGTCCACAACTCCTACAACCGTCCGGCTTACTCCCCAG
GTCACAAGACCCAACCATTCTTGCCATGGGGTGGTGGTGGTTCTGACATT
GAGTTGACTCAATCTCCATCTTCCTTGGCTGTTTCTGCTGGTGAGAAGGT
TACTATGTCTTGTAAGTCTTCCCAATCTATTTTGTACTCTTCCAACCAAA
AGAACTACTTGGCTTGGTACCAACAAAAGCCAGGTCAATCTCCAAAGTTG
TTGATTTACTGGGCTTCTACTAGAGAGTCTGGTGTTCCAGACAGATTCAC
TGGTTCTGGTTCTGGTACTGACTTCACTTTGACTATTTCTTCCGTTCAAG
CTGAGGACTTGGCTGTTTACTACTGTCACCAATACTTGTCTTCCTACACT
TTCGGTGGTGGTACTAAGTTGGAGATTAAGGGTGGTGGTGGATCTCAAGT
TCAATTGCAACAATCTGGTCCAGAGTTGGTTAGACCAGGTGCTTCTGTTA
GAATTTCTTGTAAGGCTTCTGGTTACACTTTCGCTTCTTACTACATTCAA
TGGATGAAGCAAAGACCAGGTCAAGGTTTGGAGTGGATTGGTTGGATTAA
CCCAGGTAACGTTAACACTAAGTACAACGAGAAGTTCAAGGGTAAGGCTA
CTTTGACTGCTGACAAGTCTTCCACTACCGCTTACATGCAATTGTCTTCC
TTGACTTCTGAGGACTCTGCTGTTTACTTCTGTGCTAGATCcACTTACTA
CAGACCATTGGACTACTGGGGTCAAGGTACTACCGTTACTGTTTCTTCCG
GTGGTGGTGGTTCTGGTGGTGGTGGATCCGGTGGTGGTGGTTCTGACATT
GAGTTGACTCAATCTCCATCTTCCTTGGCTGTTTCTGCTGGTGAGAAGGT
TACTATGTCTTGTAAGTCTTCCCAATCTATTTTGTACTCTTCCAACCAAA
AGAACTACTTGGCTTGGTACCAACAAAAGCCAGGTCAATCTCCAAAGTTG
TTGATTTACTGGGCTTCTACTAGAGAGTCTGGTGTTCCAGACAGATTCAC
TGGTTCTGGTTCTGGTACTGACTTCACTTTGACTATTTCTTCCGTTCAAG
CTGAGGACTTGGCTGTTTACTACTGTCACCAATACTTGTCTTCCTACACT
TTCGGTGGTGGTACTAAGTTGGAGATTAAGGGTGGTGGTGGATCTCAAGT
TCAATTGCAACAATCTGGTCCAGAGTTGGTTAGACCAGGTGCTTCTGTTA
GAATTTCTTGTAAGGCTTCTGGTTACACTTTCGCTTCTTACTACATTCAA
TGGATGAAGCAAAGACCAGGTCAAGGTTTGGAGTGGATTGGTTGGATTAA
CCCAGGTAACGTTAACACTAAGTACAACGAGAAGTTCAAGGGTAAGGCTA
CTTTGACTGCTGACAAGTCTTCCACTACCGCTTACATGCAATTGTCTTCC
TTGACTTCTGAGGACTCTGCTGTTTACTTCTGTGCTAGATCcACTTACTA
CAGACCATTGGACTACTGGGGTCAAGGTACTACCGTTACTGTTTCTTCCC
ACCACCACCACCACCAC
DT390-single-chain foldback diabody anti-human
CCR4 immunotoxin-6xHis, amino acid sequence:
AGADDVVDSSKSFVMENFASYHGTKPGYVDSIQKGIQKPKSGTQGNYDDD
WKGFYSTDNKYDAAGYSVDNENPLSGKAGGVVKVTYPGLTKVLALKVDNA
ETIKKELGLSLTEPLMEQVGTEEFIKRFGDGASRVVLSLPFAEGSSSVEY
INNWEQAKALSVELEINFETRGKRGQDAMYEYMAQACAGNRVRRSVGSSL
SCINLDWDVIRDKTKTKIESLKEHGPIKNKMSESPAKTVSEEKAKQYLEE
FHQTALEHPELSELKTVTGTNPVFAGANYAAWAVNVAQVIDSETADNLEK
TTAALSILPGIGSVMGIADGAVHHNTEEIVAQSIALSSLMVAQAIPLVGE
LVDIGFAAYNFVESIINLFQVVHNSYNRPAYSPGHKTQPFLPWGGGGSDI
ELTQSPSSLAVSAGEKVTMSCKSSQSILYSSNQKNYLAWYQQKPGQSPKL
LIYWASTRESGVPDRFTGSGSGTDFTLTISSVQAEDLAVYYCHQYLSSYT
FGGGTKLEIKGGGGSQVQLQQSGPELVRPGASVRISCKASGYTFASYYIQ
WMKQRPGQGLEWIGWINPGNVNTKYNEKFKGKATLTADKSSTTAYMQLSS
LTSEDSAVYFCARSTYYRPLDYWGQGTTVTVSSGGGGSGGGGSGGGGSDI
ELTQSPSSLAVSAGEKVTMSCKSSQSILYSSNQKNYLAWYQQKPGQSPKL
LIYWASTRESGVPDRFTGSGSGTDFTLTISSVQAEDLAVYYCHQYLSSYT
FGGGTKLEIKGGGGSQVQLQQSGPELVRPGASVRISCKASGYTFASYYIQ
WMKQRPGQGLEWIGWINPGNVNTKYNEKFKGKATLTADKSSTTAYMQLSS
LTSEDSAVYFCARSTYYRPLDYWGQGTTVTVSSHHHHHH

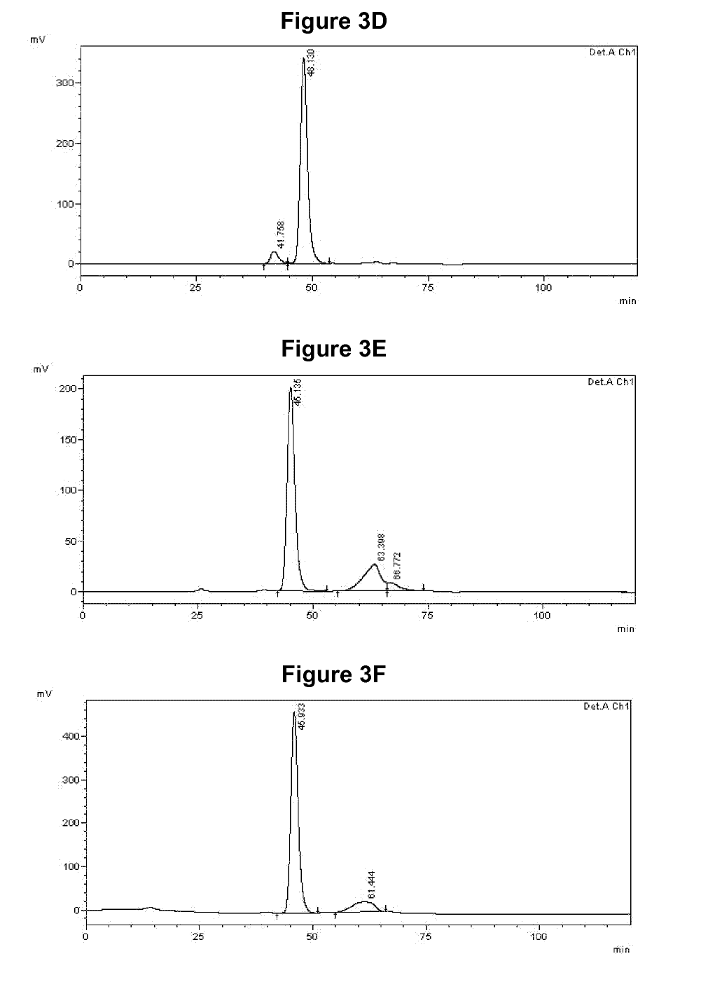

The anti-human CCR4 toxins were expressed in a unique diphtheria-toxin resistant yeast Pichia Pastoris (Liu et al., 2003) expression system using one liter Erlenmeyer flasks. The anti-human CCR4 toxins were secreted into the extracellular supernatant then captured using a Ni-sepharose fast flow resin and further purified using strong anion exchange resin. The final purification yield was ˜5 mg per liter of the original harvested supernatant for the three versions of anti-human CCR4 toxins. The purified anti-human CCR4 toxins were analyzed by SDS-PAGE (FIG. 3A) and Western blot using a mouse anti-His monoclonal antibody (FIG. 3B) and a mouse anti-diphtheria toxin monoclonal antibody (FIG. 3C) as well as HPLC analysis (FIGS. 3D-F). SDS PAGE, Western Blot, and HPLC analysis demonstrated that the three versions of the anti-human CCR4 immunotoxins were successfully expressed and purified with expected molecular weights of 70.26 kDa for the monovalent isoform, 97.57 kDa for the bivalent isoform and 96.31 kDa for the single-chain foldback diabody isoform

### Example 1.2. Binding Affinity and Blocking Analysis of the Anti-Human CCR4 Immunotoxins by Flow Cytometry

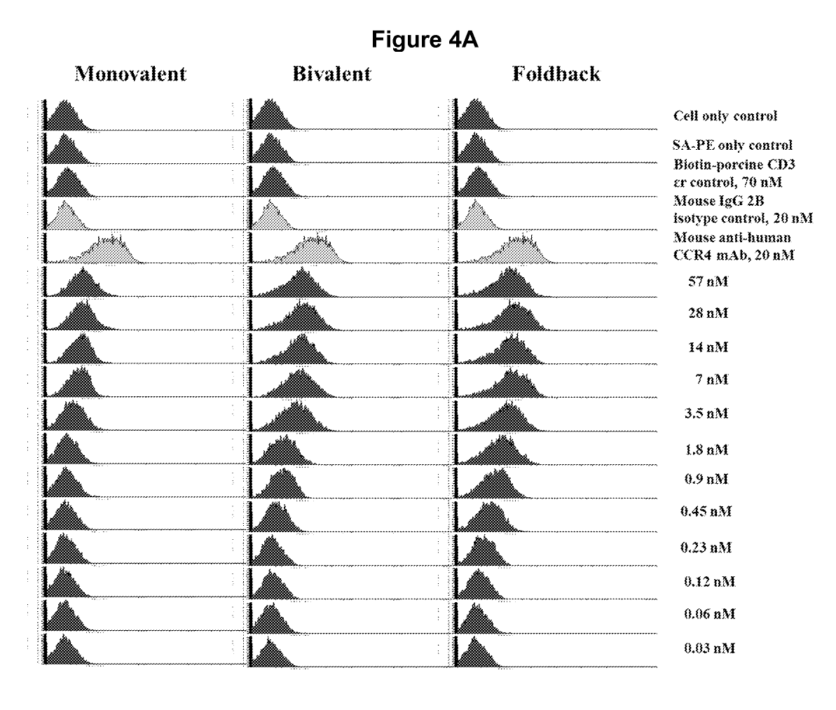

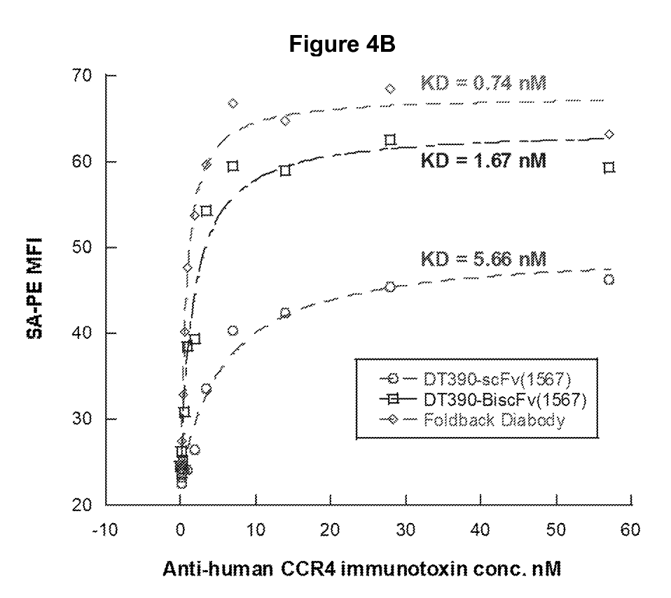

Anti-human CCR4 immunotoxins target the human CCR4⁺ cells via binding of the anti-human CCR4 scFv(1567) domain of the immunotoxins. Following the cellular internalization, the DT390 domain functions to inhibit protein synthesis resulting in the cell death (Murphy 2011). Therefore, the first critical step in determining the functionality of the anti-human CCR4 immunotoxins was to analyze their binding affinity for human CCR4. The anti-human CCR4 immunotoxins were labeled with sulfo-EZ-link NHS biotin (Thermo Scientific) for binding analysis to human CCR4⁺ CCRF-CEM leukemia cells using flow cytometry. As shown in FIG. 4A, monovalent (left panel), bivalent (middle panel) and single-chain fold-back diabody (right panel) anti-human CCR4 immunotoxins bound to the human CCR4 in a dose-dependent manner. The binding affinity was quantified by calculating the dissociation constant (Kd) for each anti-human CCR4 immunotoxin isoform from mean fluorescence intensity (MFI) (Peraino et al., 2013a). Consistent with previously developed recombinant immunotoxins (Woo et al., 2002; Kim et al., 2007; Wang et al., 2011) the bivalent isoform (Kd=1.67 nM, FIG. 4B) bound stronger than the monovalent isoform (Kd=5.66 nM, FIG. 4B) and the foldback diabody isoform was found to have the highest binding affinity (Kd=0.74 nM, FIG. 4B).

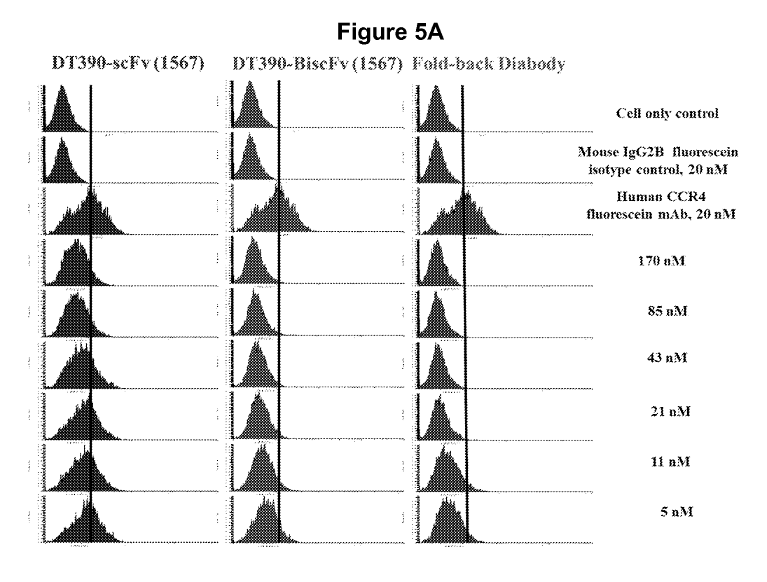

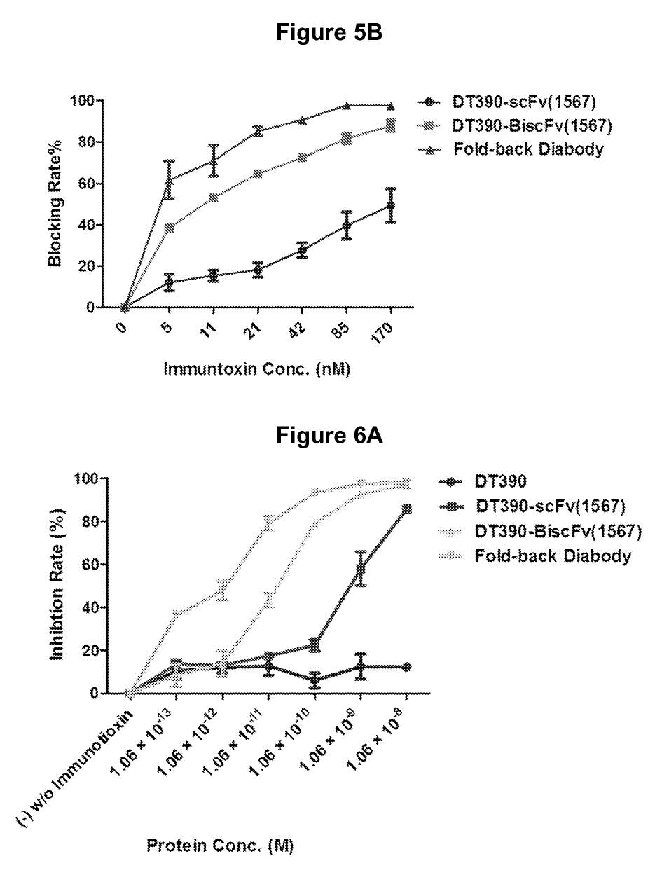

The binding specificity of the anti-human CCR4 immunotoxins were further analyzed by blocking the binding of the parent anti-human CCR4 mAb 1567 to its receptor on human CCR4+ CCRF-CEM leukemia cells. As shown in FIG. 5A, monovalent (left panel), bivalent (middle panel) and single-chain fold-back diabody (right panel) anti-human CCR4 immunotoxins blocked the binding of anti-human CCR4 mAb 1567 to the cells in a dose-dependent fashion, which suggests that the anti-human CCR4 immunotoxins binding specifically to the human CCR4 receptor. The bivalent isoform blocked the binding about 10 times more strongly than the monovalent version and the single-chain fold-back diabody is about 3 times stronger than the bivalent isoform (FIG. 5B).

To rule out the off-target effect of the immunotoxin, we analyzed three irrelevant non-human CCR4-expressing tumor cell lines with biotin-labeled foldback diabody anti-human CCR4 immunotoxin: 1) MV3; 2) M14; 3) MD-MBA-231. The results demonstrated that there is no any binding activity with these non-human CCR4-expressing tumor cell lines (data not shown). We have also analyzed the viability of these three non-human CCR4-expressing tumor cell lines by flow cytometry using propidium iodide and Annexin V following incubation with the foldback diabody anti-human CCR4 immunotoxin for 18 h. Their viabilities were not affected or minimally affected.

### Example 1.3. In Vitro Protein Synthesis Inhibition Analysis of the Anti-Human CCR4 Toxins

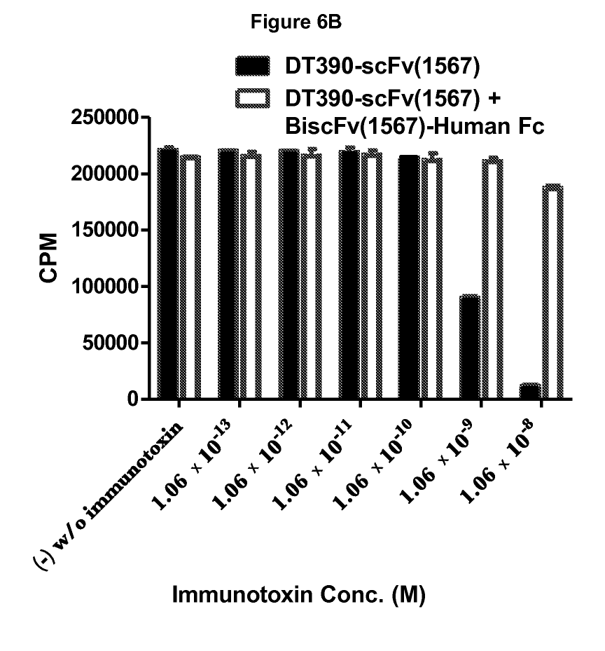

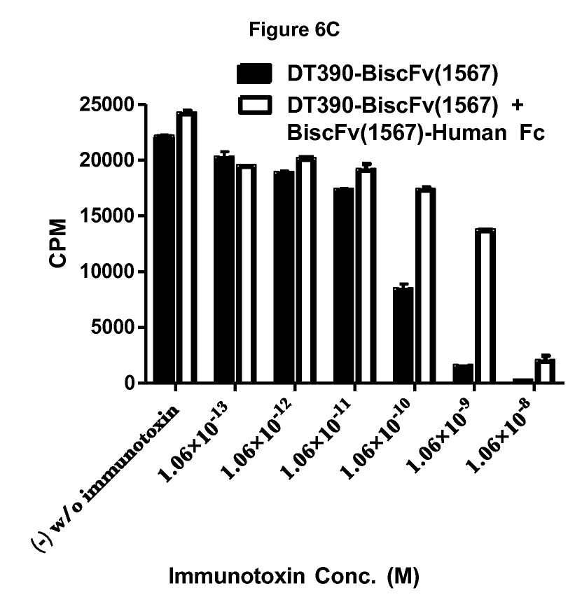

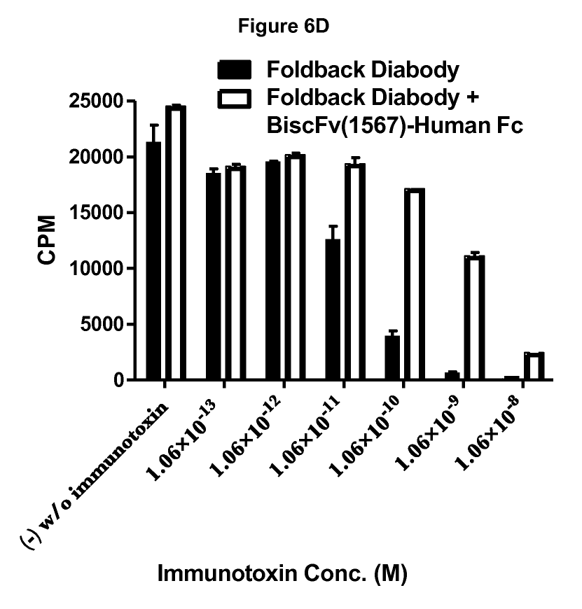

The anti-human CCR4 toxin efficacy was assessed in vitro using the protein synthesis inhibition assay through incorporating tritiated leucine. As shown in FIG. 6A, all three versions (monovalent, bivalent and single-chain foldback diabody) of anti-human CCR4 toxins are potent to inhibit the protein synthesis of human CCR4+ CCRF-CEM leukemia cells. The bivalent version (EC50=1.53×10⁻¹¹ M) is about 40 fold stronger than its monovalent counterpart (EC50=6.67×10⁻¹⁰ M). The single-chain foldback diabody version (EC50=9.42×10⁻¹³ M) is about 16 fold stronger than its bivalent counterpart and about 700 fold stronger than its monovalent isoform. The bivalent and foldback diabody strategies significantly improved the efficacy as other bivalent and foldback diabody recombinant immunotoxins (Woo et al 2002, Wang et al., 2011, Kim et al 2007).

In order to confirm that the human CCR4+ CCR-FEM leukemia cells are being targeted specifically through the interaction of the anti-human CCR4 scFv (1567) domain on the immunotoxins and the human CCR4 receptor on the cell surface, we assessed the immunotoxins' ability to halt protein synthesis in the presence of the anti-human CCR4 antibody [BiscFv (1567)-human Fc]. Target cells that were incubated with immunotoxin in the presence of the anti-human CCR4 antibody [BiscFv (1567)-human Fc] showed a marked increase in protein synthesis compared to cells which were cultured with the corresponding concentration of immunotoxin only. The anti-human CCR4 antibody [BiscFv (1567)-human Fc] acted as an inhibitor of immunotoxin as it prevented the monovalent (FIG. 6B), bivalent (FIG. 6C) and the foldback diabody (FIG. 6D) anti-human CCR4 immunotoxins from targeting the human CCR4+ cells.

### Example 1.4. In Vitro Cell Proliferation Inhibition Analysis of the Anti-Human CCR4 Toxins

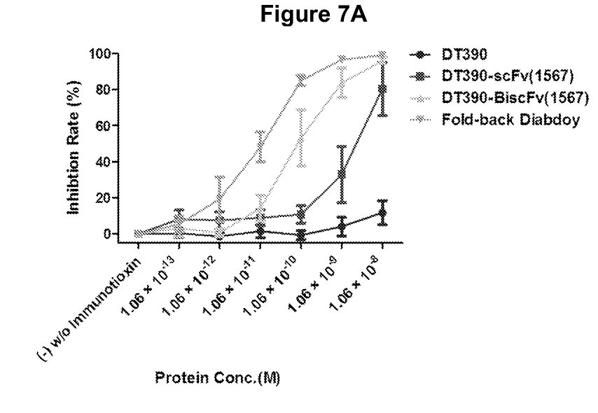

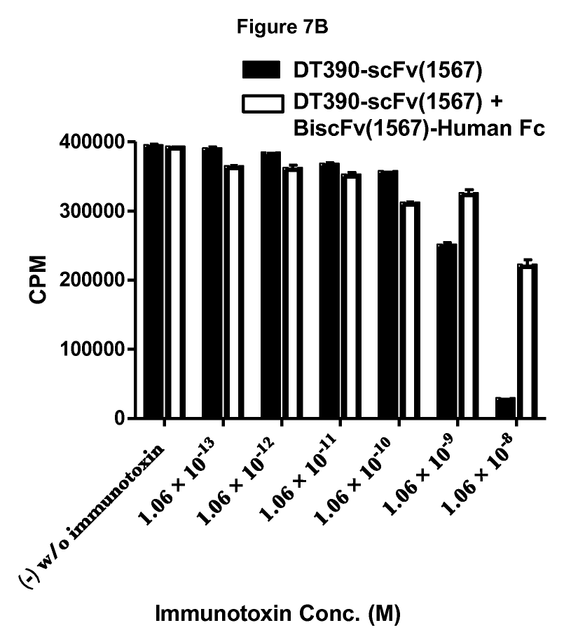

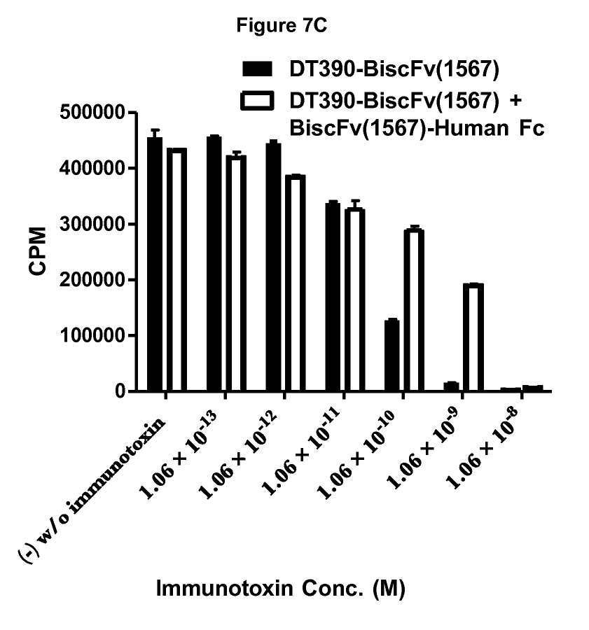

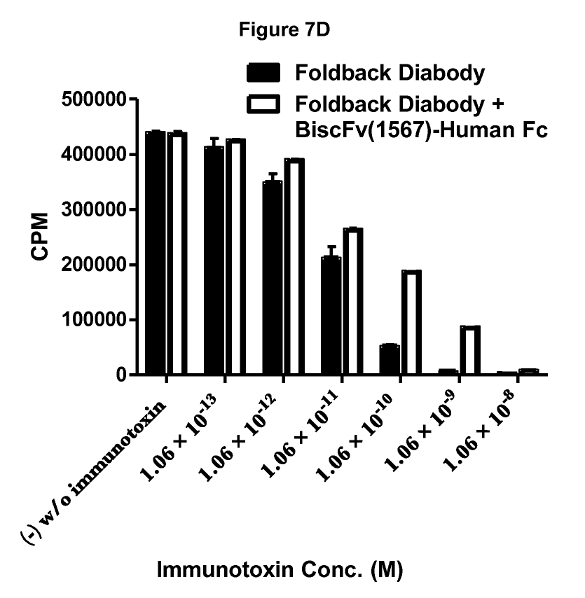

The potency of the anti-human CCR4 toxins was further assessed in vitro using a cell proliferation inhibition assay at the DNA level through incorporating tritiated thymidine. As shown in FIG. 7A, all three versions of the anti-human CCR4 toxins are potent and the bivalent isoform is about 9 fold more effective than the monovalent isoform and the foldback diabody isoform is about 20 fold more effective than the bivalent isoform and about 200 fold more effective than the monovalent isoform, which is consistent with the previous protein synthesis inhibition analysis.

Again, to double confirm the anti-human CCR4 immunotoxins bound to the target cells via interaction of the cell surface human CCR4 receptor with the anti-human CCR4 scFv (1567) domain of the immunotoxins in this cell proliferation inhibition assay, we observed the ability of the anti-human CCR4 immunotoxins to inhibit the cellular proliferation in the presence of human CCR4 inhibitor, BiscFv(1567)-human Fc. Consistently, BiscFv(1567)-human Fc drastically affected the ability of the anti-human CCR4 immunotoxins to obstruct cellular proliferation in target cells (FIG. 7B-D).

### Example 1.5. In Vitro Binding and Depletion Analysis of the Anti-Human CCR4 Immunotoxins to Human PBMC

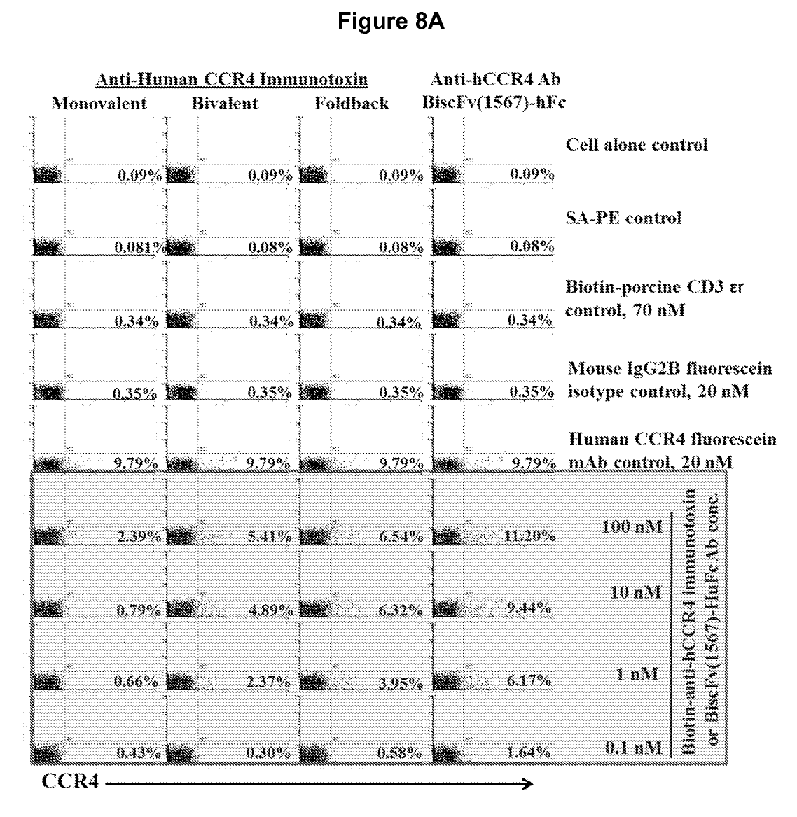

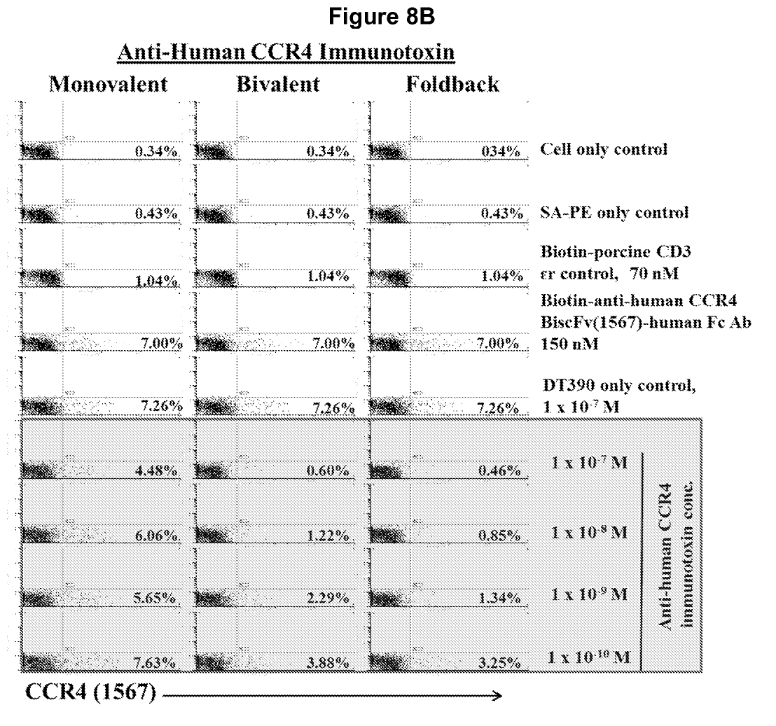

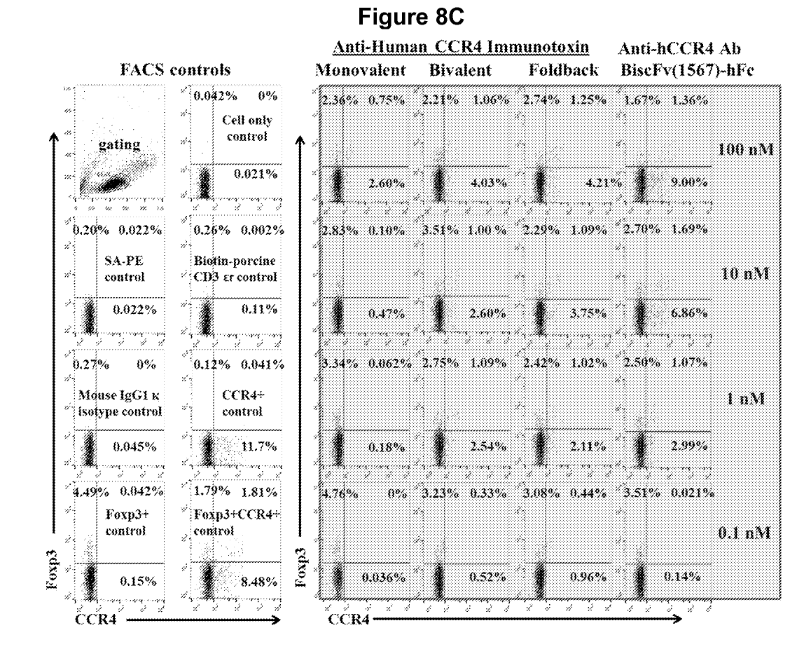

To further characterize the anti-human CCR4 immunotoxins, we performed the in vitro binding and depletion analysis of the immunotoxins to human PBMC. As shown in FIG. 8A, three versions of the biotinylated anti-human CCR4 immunotoxins bound to CCR4+ human PBMC in a dose-dependent fashion. The bivalent isoform bound stronger than the monovalent isoform and the fold-back diabody isoform is the best, which are consistent with the previous binding analysis using the human CCR4+ CCRF-CEM leukemia cell line. Based on this positive binding data, we further performed the in vitro depletion assay to the CCR4+ human PBMC using the immunotoxins. As shown in FIG. 8B, CCR4+ human PBMC was in vitro depleted in a dose dependent manner. The bivalent and fold-back diabody isoforms are better than the monovalent version and the fold-back diabody version is the best for depleting CCR4+ human PBMC. The depletion profile was double confirmed using another anti-human CCR4 mAb, PE-anti-human 194 (CCR4) mAb (Clone# L291H4, Biolegend) by flow cytometry (data not shown).

One of the main expected applications of this immunotoxin is to specifically deplete CCR4+ Tregs in vivo. Therefore we further analyzed the binding of the immunotoxins to the CCR4+ Foxp3+ human PBMC. As shown in FIG. 8C, the immunotoxins bound to the CCR4+Foxp3+ Tregs within human PBMC also in a dose dependent manner. The bivalent isoform bound stronger than the monovalent isoform and the foldback diabody isoform is the best.

To further rule out the off-target effect of the immunotoxin, we have also analyzed other human PBMC populations following incubation with the foldback diabody anti-human CCR4 immunotoxin for 48 hours: CD8 T cell (CD3+CD8+), B cell (CD19+), NK cells (CD16+CD8+) and monocyte (CD14+CD16+). The data demonstrated that there was no effect on other human PBMC populations (data not shown). In contrast, as expected, CD4 T cell (CD3+CD4+) was depleted in a dose-dependent manner as most of CCR4+ cells belong to this sub-population (data not shown).

### Example 1.6. In Vivo Efficacy Assessment of the Anti-Human CCR4 Immunotoxins Using a CCR4+ Tumor-Bearing NSG Mouse Model

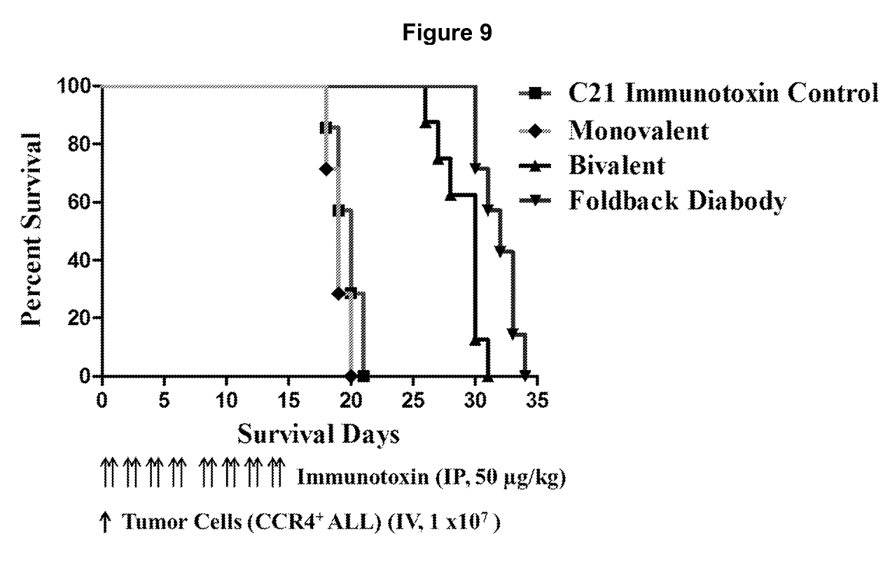

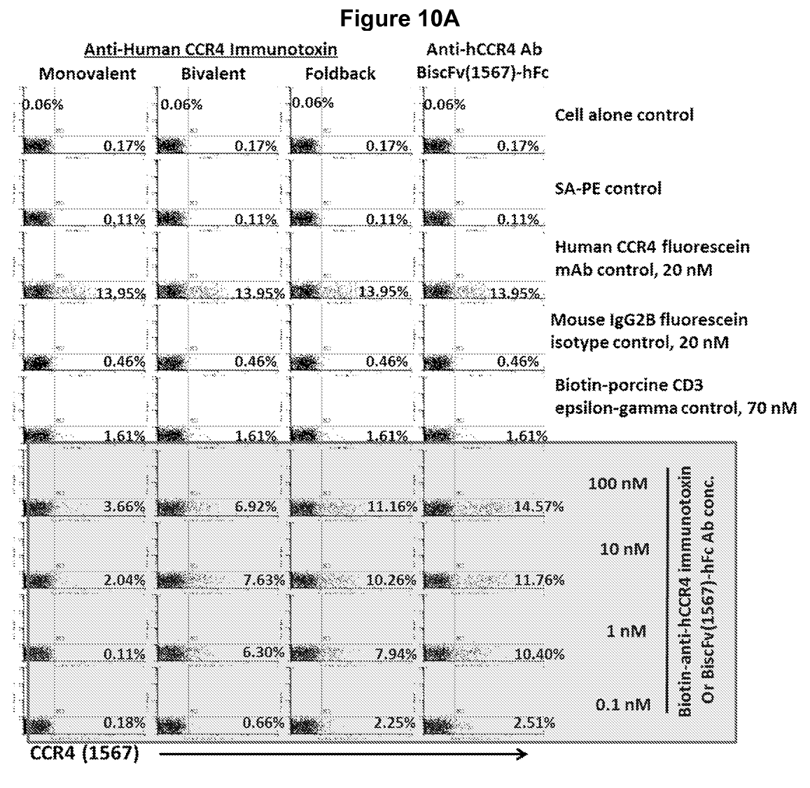

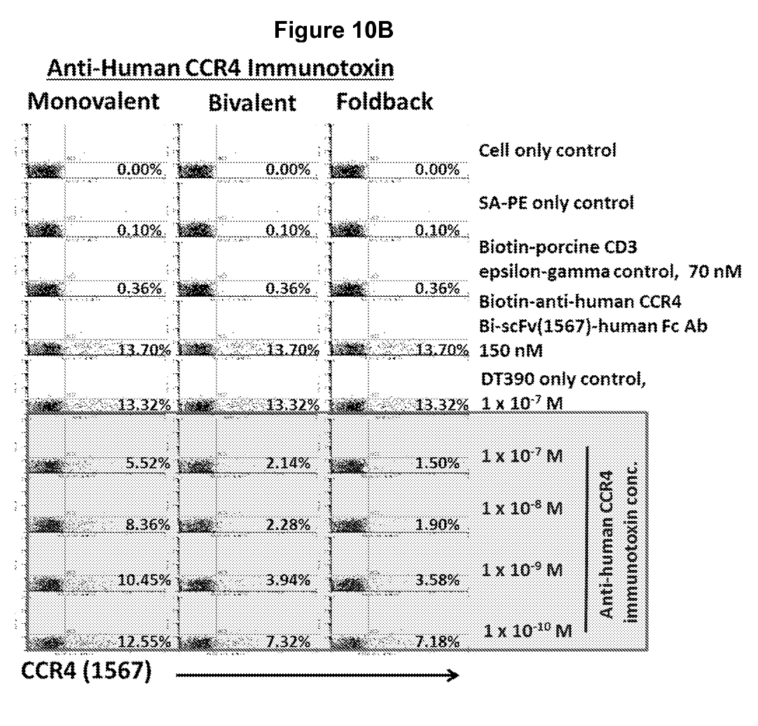

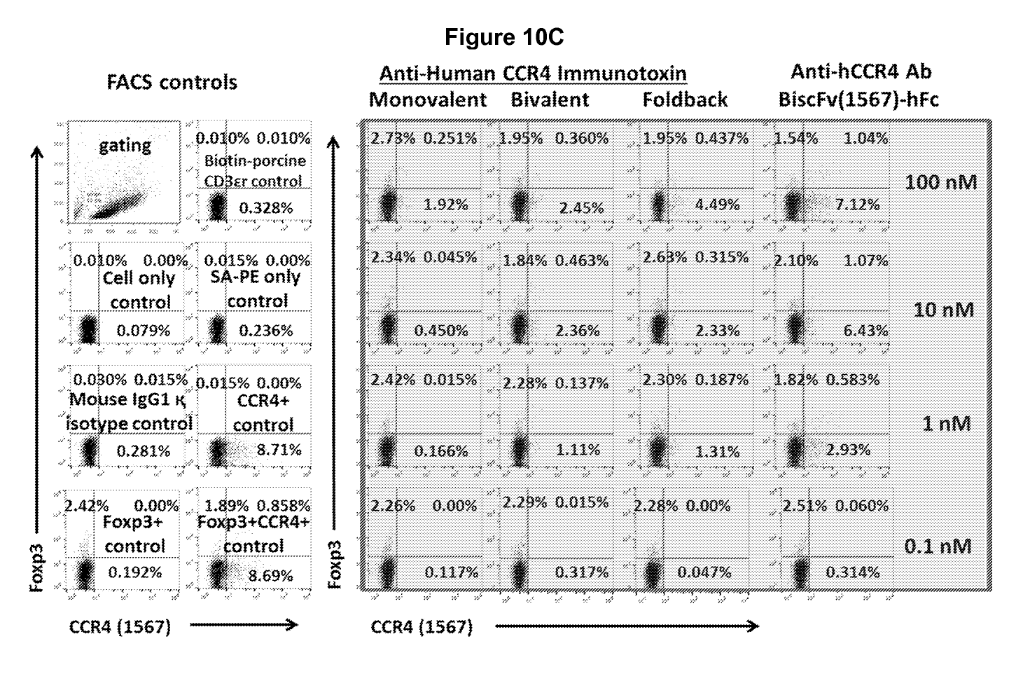

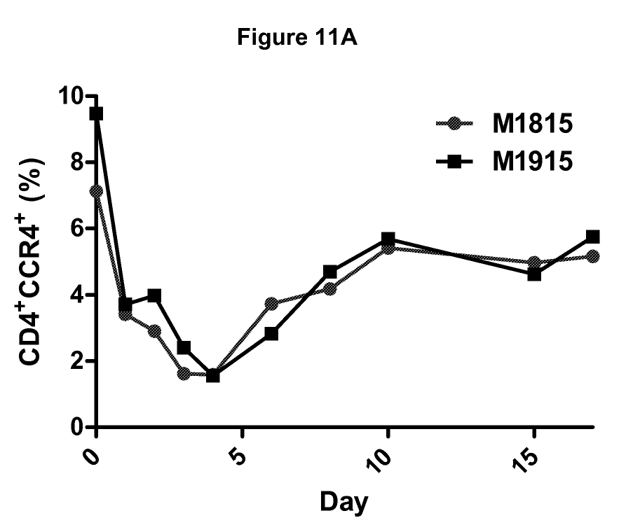

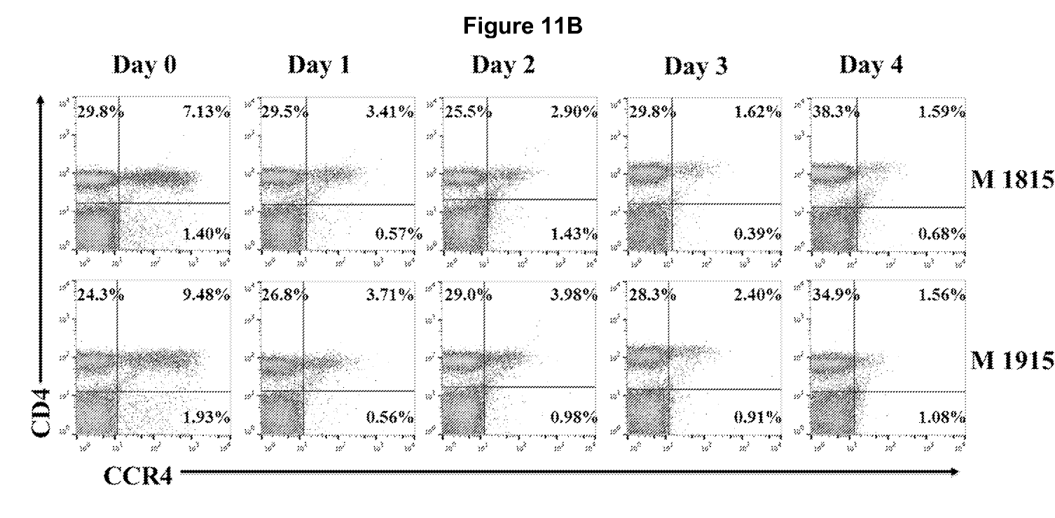

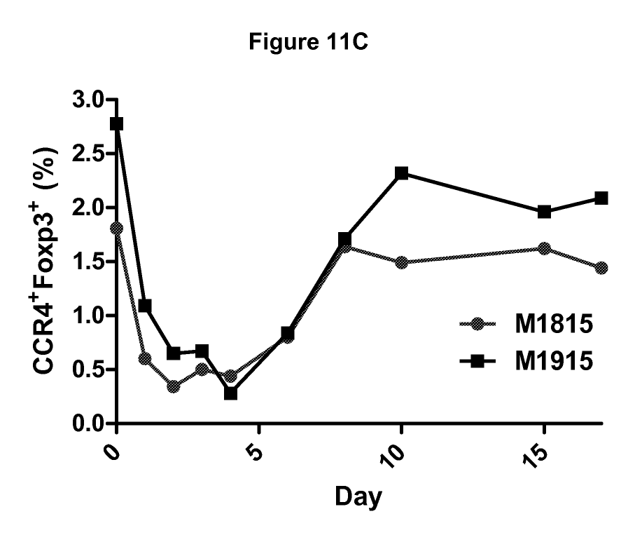

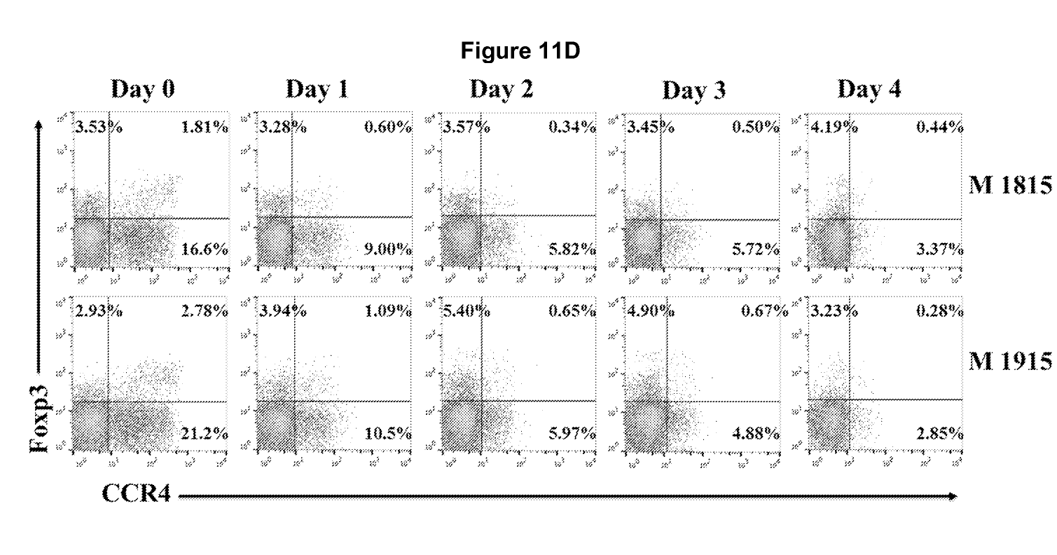

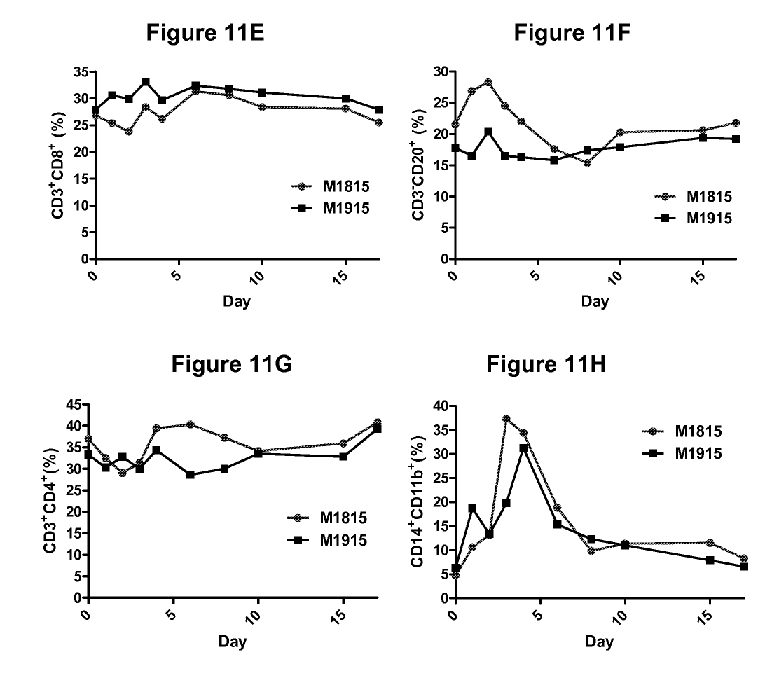

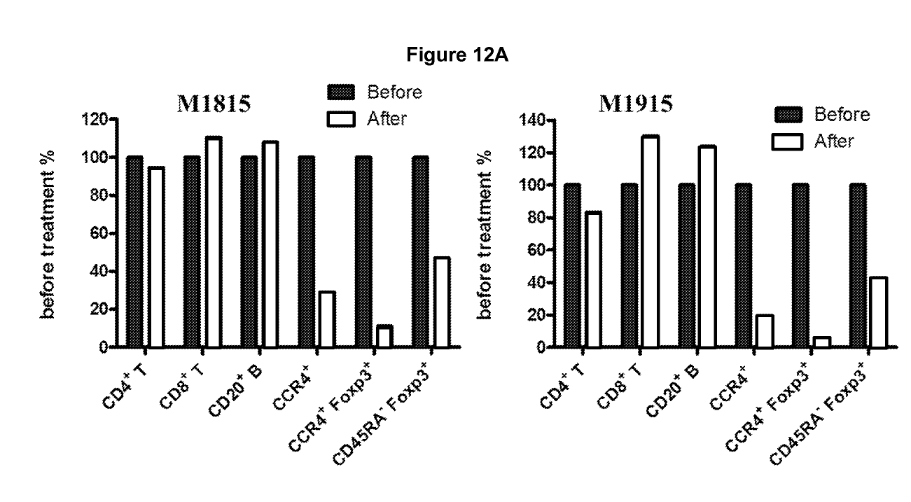

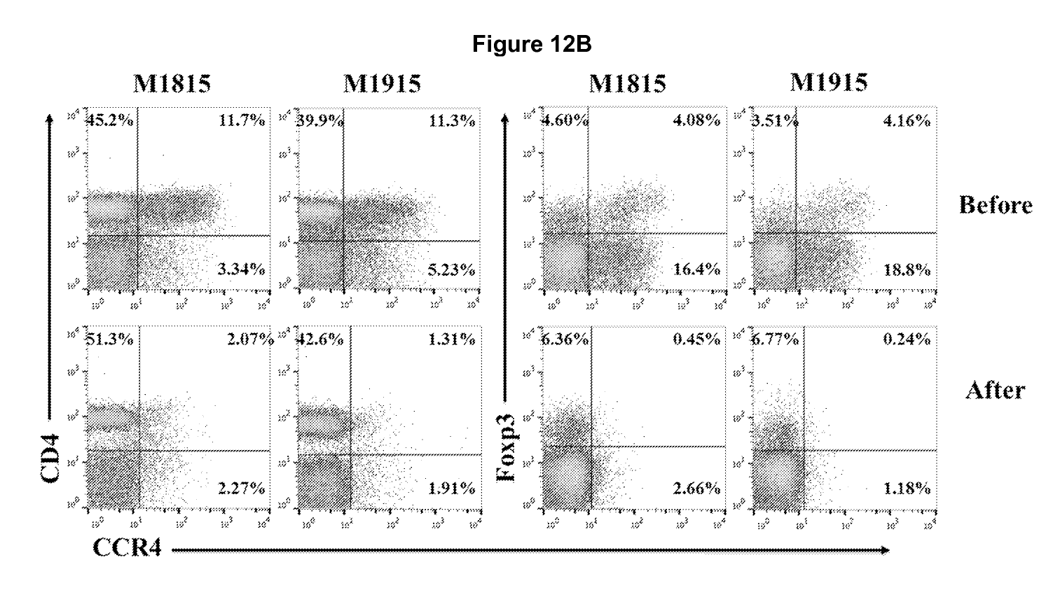

Human CCR4+ CCRF-CEM tumor-bearing NSG mouse model was used to assess the in vivo efficacy of the anti-human CCR4 immunotoxins. NSG mice were IV injected with 1×107 human CCR4+ CCRF-CEM tumor cells and treated (IP) with the anti-human CCR4 immunotoxin at 50 μg/kg BID for 4 consecutive days as one course, two course total, three day break between the two courses. This dosing schedule was based on our previous experience (Peraino et al., 2013a and 2013b) and given this CCR4+ ALL is extremely aggressive. As shown in FIG. 9, C21 immunotoxin (a non-related DT390 based immunotoxin) was injected as negative control (n=7). Both bivalent and single-chain fold-back diabody anti-human CCR4 immunotoxins significantly prolonged the animal survival from median 20 days of the negative control to 30 days using the bivalent version (n=8) and 32 days using the fold-back diabody version (n=7). The bivalent version was significantly more effective (p<0.0001) than the negative control. The fold-back diabody version is even more effective than the bivalent version (p=0.0057). The monovalent version did not prolong the animal survival with median survival time of 19 days using this dosing schedule. Mice received the anti-human CCR4 immunotoxin alone did not show any evidence of toxicity (data not shown). All animals that were injected with the human CCR4+ CCRF-CEM tumor cells succumbed to tumors, demonstrated by growth pathology and histopathology (data not shown). Human CCR4+ CCRF-CEM ALL was very aggressive to result in the animal death with only median 20 days, which speeded up our entire in vivo efficacy assessment.

### Example 2. Treg Depletion in Non-Human Primates Using a Novel Diphtheria Toxin-Based Anti-Human CCR4 Immunotoxin

In this study, we demonstrated that the anti-human CCR4 immunotoxin in vitro cross-species bound and depleted CCR4+ cells in monkey PBMC. We also demonstrated that the immunotoxin in vitro bound to the CCR4+Foxp3+ monkey Tregs. In vivo studies performed in two naive cynomolgus monkeys revealed 78-89% CCR4+Foxp3+ Treg depletion in peripheral blood lasting approximately 10 days. 89-96% CCR4+Foxp3+ Tregs in lymph nodes were also depleted. This anti-human CCR4 immunotoxin has high specificity as evidenced by minimal effects on other cell populations including CD8+ T cells, other CD4+ T cells, B cells and NK cells. To our knowledge, this is the first effective agent for depletion of non-human primate (NHP) Tregs.

Materials and Methods

The following materials and methods were used in Example 1 set forth below.

Antibodies.

FITC-anti-human CD3ε mAb (clone# SP34-2, cat#556611), PE-anti-human CD3ε mAb (clone#SP-34-2, cat#556612), PerCp-anti-human CD4 mAb (clone# L200, cat#550631), APC-anti-human CD25 mAb (clone# M-A251, cat#561399) and FITC-anti-human CD45RA (clone#5H9, cat#556626) were purchased from BD. APC-anti-human CD8 mAb (clone# RPA-T8, cat#301014), PE-CD20 (clone#2H7, cat#302306), Biotin-anti-human CD16 mAb (clone#3G8, cat#302008), FITC-anti-human CD14 mAb (clone# M5E2, cat#301803), PerCp-Cy5.5-anti-human CD11b (clone# M1/70, cat#101228), PE-anti-human CD194 (CCR4) mAb (Clone# L291H4), Alexa Fluor® 647 anti-human Foxp3 mAb (clone#150D, cat#320014) and Alex Fluor 647 Mouse IgG1 κ (clone# MOPC-21, cat#400136) were purchased from Biolegend. Human/rat CCR4 fluorescein mAb (clone 205410, cat# FAB1567F) and mouse IgG2B fluorescein isotype control (clone#133303, cat# IC0041F) were purchased from R&D Systems. BiscFv(1567)-Human Fc was produced using yeast Pichia Pastoris expression system in our lab.

In Vivo Monkey Treg Depletion.

Two male cyno monkeys (M1815: 5.1 kg, M1915: 5.3 kg) were maintained in Massachusetts General Hospital (MGH) non-human primate facility. MGH is an AAALAC accredited institute. All experiments were conducted with the approved MGH IACUC protocol (2012N000134). The foldback diabody anti-human CCR4 immunotoxin was IV bolus injected at 25 μg/kg, BID for four consecutive days, 6 hours apart. 2-3 mL of saline was injected before and after the immunotoxin injection. Sedation was performed for the immunotoxin injection and blood collection. The blood was collected daily for flow cytometry analysis in the first week and twice weekly thereafter. The animals were closely monitored twice daily during the immunotoxin injection and once daily after the immunotoxin treatment for any adverse effects. Clinical assessments for adverse events include daily clinical observation, complete blood counts and serum chemistries. The animals were weighed weekly.

Treg and other cell populations in peripheral blood were measured 3 times before the immunotoxin administration to obtain an accurate baseline. Following immunotoxin administration, peripheral blood flow cytometry were performed daily for the first week and twice weekly thereafter to monitor the effect of the immunotoxin treatment on all peripheral blood cell populations including T cells, B cells, NK cells, and monocytes. A combination of CD4, CCR4, CD45RA, Foxp3 were used to monitor the Treg populations (CCR4⁺ cell: CD4⁺CCR4⁺; CCR4⁺ Treg: CCR4⁺Foxp3⁺ among the gated CD4⁺ cells, Effector-type Treg: CD45RA⁻Foxp3⁺ among the gated CD4⁺ cells). The off-target deletion on other cell lineages was monitored by flow cytometry using antibodies against CD3, CD4, CD8, CD20, CD16, CD14 and CD11b (CD4⁺ T cell: CD3⁺CD4⁺; CD8⁺ T cell: CD3⁺CD8⁺; CD20⁺ B cell: CD3⁻CD20⁺; NK cell: CD16⁺CD8⁺; Monocyte: CD14⁺CD11b⁺).

Lymph node biopsies were performed prior to the immunotoxin injection on day −7 and after the immunotoxin administration on day 4. Treg depletion in the lymph node was monitored by flow cytometry using antibodies against CD4, CCR4, CD45RA and Foxp3 (CCR4⁺ cell: CD4⁺CCR4⁺, CCR4⁺ Treg: CCR4⁺Foxp3⁺ among the gated CD4⁺ cells, Effector-type Treg: CD45RA⁻Foxp3⁺ among the gated CD4⁺ cells). The off-target depletion in the lymph node was monitored by flow cytometry using antibodies against CD3, CD4, CD8 and CD20 (CD4 T cell: CD3⁺CD4⁺, CD8 T cell: CD3⁺CD8⁺, B cell: CD3⁻CD20⁺).

Monkey PBMC Isolation (Small Volume Blood Collection Maximal of 2 mL).

Monkey PBMC isolation was performed following BL-2 rules. 7 mL of washing buffer (1% FBS in PBS, sterile with 0.22 μM filter) was added to a 15 mL conical tube. 1-2 mL of monkey blood was added to the prepared 7 mL of washing buffer to a total of 9 mL and mixed by inverting the tube gently. 5 mL of Histopaque-1077 (Sigma, cat# H8889) was added to the bottom of a new 15 mL conical tube. The blood/washing buffer mixture was slowly overlaid on Histopaque-1077. The tube was centrifuged at 2600 rpm for 30 min with the brake off. The buffy layer was transferred to a new 15 mL conical tube and washing buffer was added to a total of 15 mL. The tube was centrifuged at 1500 rpm for 10 min with the brake on. The supernatant was decanted and the cells were loosened gently by taping the tube wall. 4.5 mL of pure water (HyClone cell culture grade pure water, Thermo, cat# SH30529.03) was added to the cells and mixed by pipetting up and down. 0.5 mL of 10×DPBS (Cellgro, cat#20-031-CV) was immediately (within 10 seconds) added and mixed by inverting. The tube was centrifuged at 1500 rpm for 10 min with the brake on. The supernatant was decanted and the remaining supernatant was carefully removed with pipette. The pellet was loosened by tapping the tube wall. 3-5 mL of the washing buffer was added and filtered with a 40 μM cell strainer (Corning, cat#431750). The cells were counted with trypan blue. Monkey PBMC isolation from big volume blood, in vitro binding and depletion analysis of the anti-human CCR4 immunotoxins to human CCR4 on monkey PBMC using flow cytometry was performed as previously described ¹.

Monkey Whole Blood Flow Cytometry Analysis.

The monkey whole blood staining for flow cytometry analysis was performed following BL-2 rules. 100 μL of the heparinized monkey blood was added into each flow cytometry tube. 2 mL of the FACS buffer (1× Hanks Balanced Salt Solution with Ca⁺ and Mg⁺, 0.1% Bovine serum albumin and 0.1% sodium azide) was added into the tube and mixed by vortex. The tube was spun down at 1200 rpm for 5 min in room temperature. The supernatant was discarded gently and washed once more using the FACS buffer as above. 10 μL of the conjugated antibody was added to the tube and mixed by gentle vortex. The tube was covered with tin foil and incubated at 4° C. for 30 min. The cells were washed twice with 2 mL of the FACS buffer at 1200 rpm for 5 min in room temperature. The supernatant was discarded and suspended by ratcheting. 2 mL of 1×BD FACS lysing buffer (BD BioSciences, cat#349202) was added into the tube and capped. The tube was mixed by vortex and incubated for 15 min. The tube was mixed by vortex and spun down at 1200 rpm for 5 min in room temperature. The cells were washed twice again with the FACS buffer. 400 μL of the FACS buffer was added into the tube and stored at 4° C. in the dark until running on the flow cytometry machine.

Foxp3 Flow Cytometry Analysis for Monkey PBMC.

Monkey PBMC was re-suspended at 1×10⁷ cells/mL in cold FACS buffer (lx Hanks Balanced Salt Solution with Ca⁺ and Mg⁺, 0.1% Bovine serum albumin and 0.1% sodium azide). 100 μL of the cell suspension was aliquoted into each tube (1×10⁶ cells). The surface staining was performed as normal surface staining procedure. The cells was washed twice using cold FACS buffer. The cells were spun down and the supernatant was poured off. The pellet was loosened with pulse vortex. 1 mL of fresh 1× fixation/permeabilization working solution (4× fixation/permeabilization concentrate, eBioscience, cat#5123-43) was added and mixed by pulse vortex. The tube was incubated for 30 min to 45 min at 4° C. in the dark. 2 mL of the fresh 1× permeabilization buffer (10× permeabilization buffer, eBioscience, cat#8333-56) was added into the tube and spun down. The supernatant was poured off and washed once more. The conjugated anti-Foxp3 mAb or isotype control was added into the tube and incubated at 4° C. in the dark for at least 30 min. The cells were washed twice with fresh 1× permeabilization buffer. 300 μL of FACS buffer was added and stored at 4° C. until running the flow cytometry machine.

### Example 2.1 Depletion of CCR4+ Cells in a Non-Human Primate

To investigate whether these immunotoxins can cross-species react with NHP CCR4+ PBMC, we performed in vitro binding and depletion assays. All three versions of the biotinylated anti-human CCR4 immunotoxins, monovalent, bivalent and fold-back diabody, bound to CCR4+ monkey PBMC in a dose-dependent fashion with the diabody isoform demonstrating the strongest binding affinity, followed by the bivalent isoform and then the monovalent isoform (FIG. 1A). These results are consistent with the previous binding analysis performed with human CCR4+ CCRF-CEM leukemia cell line and human PBMC 1. In vitro depletion assay also demonstrated a dose dependent depletion of CCR4+ monkey PBMC with the fold-back diabody version showing the most efficacy (FIG. 1B). Depletion was confirmed using another anti-human CCR4 mAb, PE-anti-human CD194 (CCR4) mAb (Clone# L291H4, Biolegend) by flow cytometry. Next, the binding affinity of the immunotoxins to CCR4+ Foxp3+ Tregs in monkey PBMC was assessed. The immunotoxins bound to the CCR4+Foxp3+ Tregs within monkey PBMC in a dose dependent manner, and again with the fold-back diabody isoform showing stronger binding compared to the other two isoforms (FIG. 1C). These in vitro data prompted us to test in vivo Treg depletion function of the immunotoxins in nonhuman primates.

In vivo depletion studies were performed in two naive cynomolgus monkeys. Based on the results of previous experiments 1, the fold-back diabody anti-human CCR4 immunotoxin was chosen for the in vivo monkey Treg depletion experiments. The anti-human CCR4 immunotoxin was administered intravenously at a dose of 25 μg/kg, twice daily, 6 hours apart for 4 consecutive days. This dosing strategy was chosen based on our previous experience with another recombinant diphtheria toxin based immunotoxin that targets CD3+ T cells in NHP [A-dmDT390-scfbDb(C207)] (Kim, G. B. et al. A fold-back single-chain diabody format enhances the bioactivity of an anti-monkey CD3 recombinant diphtheria toxin-based immunotoxin. Protein Eng. Des. Sel. 20, 425-432 (2007)). The CD3 immunotoxin was constructed in a similar fashion as the CCR4 immunotoxin with the DT390 domain being identical for both. This dosing strategy using CD3 immunotoxin demonstrated efficacy while showing minimal toxicity 3-4. Depletion of Tregs was monitored by flow cytometry. With the described dosing strategy, up to 80% depletion of monkey CCR4+ cells in the peripheral blood was achieved and the depletion lasted for approximately 10 days (FIGS. 2A-B). 78-89% of CCR4+Foxp3+ Tregs in the peripheral blood were depleted with similar duration (Figs. C-D). Other cell populations including CD8+ T cells, other CD4+ T cells, B cells and NK cells were unaffected or minimally affected (FIGS. 2E-G and data not shown). The CD4+ cell population was minimally affected as CCR4+ Tregs and CCR4+Th2 cells only occupy a small fraction of the entire CD4+ cell population. Remarkably 82-88% of monkey CCR4+ cells and 89-96% of CCR4+Foxp3+ monkey Tregs were also depleted from the lymph nodes after the four-day course of immunotoxin (FIG. 3a-b). Most of the depleted lymph node CCR4+Foxp3+ Tregs belongs to CD45RA-Foxp3+ effector Tregs (FIG. 3a). Consistent with the peripheral blood analysis other cell populations (CD8+ T cells, CD20+ B cells and other CD4+ T cells) in the lymph nodes were not affected (FIG. 3a). Of note, since our immunotoxin was constructed using anti-human CCR4 scFv (1567) 1, PE-anti-human CD194 (CCR4) mAb (Clone# L291H4, Biolegend) was used for all of the CCR4+ cell analysis by flow cytometry.

Clinically the animals were healthy without any adverse effects from the immunotoxin for the entire duration of the study. Transient decreased appetite was observed and was most likely due to procedural sedations as the appetite loss was also observed during sedation for blood draw without medication administration. We speculate that there remains room for dose escalation and increased duration, which may improve the Treg depletion further. Additional experiments are necessary to optimize the dosing regimen. We also speculate that the partial toxicity of the diphtheria toxin based anti-CD3 immunotoxins 3, 6, 7 and IL-2 fusion toxins 8, Ontak® may have resulted from stimulation of CD3 or CD25 receptors by the anti-CD3 scFv or IL-2 domains. In contrast, no stimulation effect was observed due to the anti-human CCR4 scFv domain of the anti-human CCR4 immunotoxin despite carrying the same DT390 domain.

Interestingly, our CCR4 immunotoxin had an inverse effect on the monocyte population, which was elevated correlating with CCR4+ Foxp3+ Treg depletion (FIG. 2H). This observation suggests that monkey CCR4+ Tregs may directly suppress the maturation of monocyte similar to the previously reported effect of CCR4+ Tregs on suppression of dendritic cell (DC) maturation 9. We did not monitor DC in peripheral blood or monocyte and DC in the lymph node samples. We speculate that both monkey monocyte and DC were elevated due to Treg depletion. Further characterization of the effects on monocyte and DC using this unique CCR4 immunotoxin may shed light on the interaction between Tregs and maturation of important antigen-presenting cell populations.

In summary, NHP CCR4+Foxp3+ Tregs were successfully depleted in vivo from both peripheral blood and lymph nodes using a novel fold-back diabody anti-human CCR4 immunotoxin. To the best our knowledge, this is the first effective agent for NHP Treg depletion in vivo. The results of this study show that this anti-human CCR4 immunotoxin will show similar efficacy in depleting human CCR4+Foxp3+ Tregs, which implies potential clinical applications for directly targeting CCR4+ tumors and as an indirect immunotherapy for other advanced malignancy.

### REFERENCES

Bayry, J., Tartour, E., Tough, D. F., 2014, Targeting CCR4 as an emerging strategy for cancer therapy and vaccines. Trends Pharmacol Sci. 35, 163.
Bayry, J., Tchilian, E. Z., Davies, M. N., Forbes, E. K., Draper, S. J., Kaveri, S. V., Hill, A. V., Kazatchkine, M. D., Beverley, P. C., Flower, D. R., Tough, D. F., 2008, In silico identified CCR4 antagonists target regulatory T cells and exert adjuvant activity in vaccination. Proc Natl Acad Sci USA 105, 10221-6.
Chang, D. K., Sui, J., Geng, S., Muvaffak, A., Bai, M., Fuhlbrigge, R. C., Lo, A., Yammanuru, A., Hubbard, L., Sheehan, J., Campbell, J. J., Zhu, Q., Kupper, T. S., Marasco, W. A., 2012, Humanization of an anti-CCR4 antibody that kills cutaneous T-cell lymphoma cells and abrogates suppression by T-regulatory cells. Mol Cancer Ther. 11, 2451.
Davies, M. N., Bayry, J., Tchilian, E. Z., Vani, J., Shaila, M. S., Forbes, E. K., Draper, S. J., Beverley, P. C., Tough, D. F., Flower, D. R., 2009, Toward the discovery of vaccine adjuvants: coupling in silico screening and in vitro analysis of antagonist binding to human and mouse CCR4 receptors. PLoS One 4, e8084.
Endo, Y., Hirahara, K., Yagi, R., Tumes, D., Nakayama, T. 2014. Pathogenic memory type Th2 cells in allergic inflammation. Trends Immunol. 35(2): 69-78.
Ferenczi, K., Fuhlbrigge, R C., Pinkus, J., Pinkus G S., Kupper, T S., 2002. Increased CCR4 expression in cutaneous T cell lymphoma. 119(6): 1405-1410.
Holliger, P., Prospero, T., Winter, G. 1993 “Diabodies”: Small bivalent and bispecific antibody fragments, 90:6444-6448
Honjo, A., Ogawa, H., Azuma, M., Tezuka, T., Sone, S., Biragyn, A., Nishioka, Y., 2013, Targeted reduction of CCR4⁺ cells is sufficient to suppress allergic airway inflammation. Respir Investig. 51, 241.
Ishida, T. and Ueda R., 2006, CCR4 as a novel molecular target for immunotherapy of cancer. Cancer Sci. 97(11): 1139-1146.
Kim, G. B., Wang, Z., Liu, Y. Y., Stavrou, S., Mathias, A., Goodwin, K. J., Thomas, J. M., and Neville Jr., D. M., 2007, A fold-back single-chain diabody format enhances the bioactivity of an anti-monkey CD3 recombinant diphtheria toxin-based immunotoxin. Protein Eng. Des. Sel. 20, 425.
Liu, Y. Y., Woo, J. H., and Neville Jr., D. M., 2003, Targeted introduction of a diphtheria toxin resistant mutation into the chromosomal EF-2 locus of Pichia Pastoris and expression of immunotoxin in the EF-2 mutants. Protein Expr Purif. 30, 262.
Mikhak, Z., Fukui, M., Farsidjani, A., Medoff, B. D., Tager, A. M., and Luster, A. D. 2009. Contribution of CCR4 and CCR8 to antigen-specific TH2 cell trafficking in allergic pulmonary inflammation. J Allergy Clin Immunol 123:67.
Murphy, J. R., 2011, Mechanism of Diphtheria Toxin Catalytic Domain Delivery to the Eukaryotic Cell Cytosol and the Cellular Factors that Directly Participate in the Process. Toxins 3, 294.
Nishikawa, H., Sakaguchi, S., 2014, Regulatory T cells in cancer immunotherapy. Curr Opin Immunol. 27, 1-7.
Panina-Bordignon, P., Papi, A., Mariani, M., Di Lucia, P., Casoni, G., Bellettato, C., Buonsanti, C., Miotto, D., Mapp, C., Villa, A., Arrigoni, G., Fabbri, L., Sinigaglia, F., 2001. The C—C chemokine receptors CCR4 and CCR8 identify airway T cells of allergen-challenged atopic asthmatics. J Clin Invest. 107(11): 1357-1364.
Peraino, J. S., Hermanrud, C. E., Springett, L., Zhang, H., Li, G., Srinivasan, S., Gusha, A., Sachs, D. H., Huang, C. A., and Wang, Z., 2012, Expression and characterization of recombinant soluble porcine CD3 ectodomain molecules: mapping the epitope of an anti-porcine CD3 monoclonal antibody 898H2-6-15. Cell Immunol. 276, 162.
Peraino, J. S., Schenk, M., Zhang, H., Li, G., Hermanrud, C. E., Neville Jr. D. M., Sachs, D. H., Huang, C. A., Duran-Struuck, R., and Wang, Z., 2013a, A truncated diphtheria toxin based recombinant porcine CTLA-4 fusion toxin. J Immunol Methods 391, 103.
Peraino, J. S., Schenk, M., Li, G., Zhang, H., Farkash, E. A., Sachs, D. H., Huang, C. A., Duran-Struuck, R., Wang, Z., 2013b, Development of a diphtheria toxin-based recombinant porcine IL-2 fusion toxin for depleting porcine CD25+ cells. J Immunol Methods 398-399, 33.
Peraino, J. S., Zhang, H., Rajasekera, P. V., Wei, M., Madsen, J. C., Sachs, D. H., Huang, C. A., Wang, Z., 2014, Diphtheria toxin-based bivalent human IL-2 fusion toxin with improved efficacy for targeting human CD25+ cells. J Immunol Methods. 405, 57.
Schuh, J. M., Power, C., Proudfoot, A. E., Kunkel, S. L., Lukacs, N. W., and Hogaboam, C. M. 2002. Airway hyper responsiveness, but not airway remodeling, is attenuated during chronic pulmonary allergic responses to Aspergillus in CCR4−/− mice. FASEB J 16:1313.
Pere, H., Tanchot, C., Bayry, J., Terme, M., Taieb, J., Badoual, C., Adotevi, O., Merillon, N., Marcheteau, E., Quillien, V. R., Banissi, C., Carpentier, A., Sandoval, F., Nizard, M., Quintin-Colonna, F., Kroemer, G., Fridman, W. H., Zitvogel, L., Oudard, S. P., Tartour, E., 2012, Comprehensive analysis of current approaches to inhibit regulatory T cells in cancer. Oncoimmunology 1, 326-333.
Pere, H., Montier, Y., Bayry, J., Quintin-Colonna, F., Merillon, N., Dransart, E., Badoual, C., Gey, A., Ravel, P., Marcheteau, E., Batteux, F., Sandoval, F., Adotevi, O., Chiu, C., Garcia, S., Tanchot, C., Lone, Y. C., Ferreira, L. C., Nelson, B. H., Hanahan, D., Fridman, W. H., Johannes, L., Tartour, E., 2011, A CCR4 antagonist combined with vaccines induces antigen-specific CD8+ T cells and tumor immunity against self antigens. Blood 118, 4853-62.
Sugiyama, D., Nishikawa, H., Maeda, Y., Nishioka, M., Tanemura, A., Katayama, I., Ezoe, S., Kanakura, Y., Sato, E., Fukumori, Y., Karbach, J., Jäger, E., Sakaguchi, S., 2013, Anti-CCR4 mAb selectively depletes effector-type FoxP3+CD4+ regulatory T cells, evoking antitumor immune responses in humans. Proc Natl Acad Sci USA. 110, 17945.
Viney, J. M., Andrew, D. P., Phillips, R. M., Meiser, A., Patel, P., Lennartz-Walker, M., Cousins, D. J., Barton, N. P., Hall, D. A., Pease, J. E., 2014, Distinct conformations of the chemokine receptor CCR4 with implications for its targeting in allergy. J Immunol. 192, 3419.
Vitali, C., Mingozzi, F., Broggi, A., Barresi, S., Zolezzi, F., Bayry, J., Raimondi, G., Zanoni, I., Granucci, F., 2012, Migratory, and not lymphoid-resident, dendritic cells maintain peripheral self-tolerance and prevent autoimmunity via induction of iTreg cells. Blood 120, 1237-45.
Wang, Z., Duran-Struuck, R., Crepeau, R., Matar, A., Hanekamp, I., Srinivasan, S., Neville, D. M., Sachs D. H., and Huang C. A., 2011, Development of a Diphtheria Toxin Based Antiporcine CD3 Recombinant Immunotoxin. Bioconjug Chem. 22, 2014.
Wei, M., Marino, J., Trowell, A., Zhang, H., Peraino, J. S., Priyani V. Rajasekera, P. V., Madsen, J. C., Sachs, D. H., Huang, C. A., Benichou, G., Wang, Z., 2014, Diphtheria toxin-based recombinant murine IL-2 fusion toxin for depleting murine regulatory T cells in vivo. Protein Eng Des Sel. (PEDS). 27: 289-295.
Woo, J. H., Liu, Y. Y., Mathias, A., Stavrou, S., Wang, Z., Thompson, J., and Neville Jr. D. M., 2002, Gene optimization is necessary to express a bivalent anti-human anti-T cell immunotoxin in Pichia Pastoris. Protein Expr. Purif. 25, 270.
Yamada, Y., A. Aoyama, G. Tocco, S. Boskovic, O. Nadazdin, A. Alessandrini, J. C. Madsen, A. B. Cosimi, G. Benichou, and T. Kawai. 2012. Differential effects of denileukin diftitox IL-2 immunotoxin on NK and regulatory T cells in nonhuman primates. J Immunol 188: 6063-6070.
Yoshie, O., Fujisawa, R., Nakayama, T., Harasawa H., Tago, H., Izawa, D., Hieshima, K., Tatsumi, Y., Matsushima, K., Hasegawa, H., Kanamaru, A., Kamihira, S., Yamada, Y., 2002. Frequent expression of CCR4 in adult T-cell leukemia and human T-cell leukemia virus type 1-transformed T cells. Blood, 99(5).
Yoshie, O. and Matsushima, K., 2014. CCR4 and its ligands: from bench to bedside. Int Immunol. epub Aug. 2, 2014.
Zou, W., 2006 Regulatory T cells, tumour immunity and immunotherapy. Nat Rev Immunol. 6, 295.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An anti-human chemokine (C—C motif) Receptor 4 (CCR4) immunotoxin comprising a first part comprising a cytotoxic protein linked to a second part comprising at least one human CCR4-binding domain, optionally with an intervening linker between the first and second parts.

2. The immunotoxin of claim 1, wherein the second part comprises at least two human CCR4-binding domains.

3. The immunotoxin of claim 1, comprising an intervening linker between the first and second parts.

4. The immunotoxin of claim 2, wherein the at least two human CCR4-binding domains are joined by one or more linkers.

5. The immunotoxin of claim 3, wherein the linker comprises four glycines and a serine residue (GGGGS (SEQ ID NO:16).

6. The immunotoxin of claim 1, wherein the human CCR4-binding domain comprises an antigen-binding portion of an anti-human CCR4 antibody.

7. The immunotoxin of claim 6, wherein the antigen-binding portion of an anti-human CCR4 antibody comprises V_{H} and V_{L} regions from an anti-human CCR4 antibody.

8. The immunotoxin of claim 7, wherein the V_{H} and V_{L} regions are from Mab1567 (clone 205410).

9. The immunotoxin of claim 7, wherein the human CCR4-binding domain is a monovalent ScFv, and optionally wherein the V_{H} and V_{L} regions are linked by a linker of 1-50 amino acids.

10. The immunotoxin of claim 7, wherein the human CCR4-binding domain is a diabody, and the V_{H} and V_{L} regions are linked by a linker of 1-5 amino acids.

11. The immunotoxin of claim 1, wherein the human CCR4 binding domain comprises SEQ ID NO:7 or SEQ ID NO:9.

12. A nucleic acid encoding the immunotoxin of claim 1.

13. The nucleic acid of claim 12, which is codon optimized for expression in Pichia Pastoris.

14. A vector comprising the nucleic acid of claim 12.

15. A method of depleting CCR4+ FOXP3^{hi}CD45RA⁻CD25^{hi} Tregs in a subject, the method comprising administering a therapeutically effective amount of the immunotoxin of claim 1.

16. A method of treating a subject who has a disease associated with CCR4⁺ Treg cells, CCR4+ tumor cells, or CCR+ Th2 cells, the method comprising administering to the subject a therapeutically effective amount of the immunotoxin of claim 1.

17. The method of claim 15, wherein the disease is cancer.

18. The method of claim 17, wherein the cancer is associated with CCR4⁺ Treg cells.

19. The method of claim 17, wherein the cancer is a solid tumor.

20. The method of claim 17, wherein the cancer is a carcinoma, sarcoma, or melanoma.

21. The method of claim 17, wherein the cancer is associated with CCR4+ tumor cells.

22. The method of claim 17 wherein the cancer is skin homing cutaneous T cell lymphoma, adult T cell leukemia/lymphoma, or acute T-cell lymphoblastic leukemia, cutaneous T cell lymphoma/leukemia, anaplastic large cell lymphoma, peripheral T cell lymphoma; and adult T-cell leukemia/lymphoma.

23. The method of claim 15 wherein the disease is caused by allergic inflammation.

24. The method of claim 23, wherein the disease caused by allergic inflammation is asthma.
